(19) United States
(12) Patent Application Publication
(10) Pub. No.: US 2026/0301173 A1
(43) Pub. Date: Oct. 1, 2026

(54) AI-BASED METHOD AND SYSTEM FOR DETECTION OF ABNORMAL CELLS IN URINE

(71) Applicant: VisionGate, Inc., Woodinville, WA (US)

(72) Inventors: Michael G. Meyer, Phoenix, AZ (US); Alan Nelson, Woodinville, WA (US)

(21) Appl. No.: 19/631,937

(22) Filed: Mar. 27, 2026

Related U.S. Application Data
(60) Provisional application No. 63/779,999, filed on Mar. 28, 2025.

(51) Int. Cl. G06T 7/00; G06V 10/764; G06V 20/69; G16H 10/40; G16H 50/20; G16H 50/30

(52) U.S. Cl. CPC G06T 7/0012; G06V 10/764; G06V 20/698; G16H 10/40; G16H 50/20; G16H 50/30; G06T 2207/10101; G06T 2207/30024; G06V 2201/03

(57) ABSTRACT

The present disclosure provides an artificial intelligence (AI)-based urine cell detection method including generating, for a plurality of objects in a urine sample and by the optical tomography system, a 3D image of an object from the urine sample, and evaluating the 3D image using 3D AI-based classification to determine if the object is an abnormal cell or an object other than an abnormal cell. The disclosure further provides optical coherence tomography (OCT) systems able to perform this and other methods of abnormal cell classifications from a urine sample.

### TECHNICAL FIELD

The present disclosure relates to a system and method for classification of cells in urine to detect abnormal cells. The system and method may also detect white blood cells, crystals, red blood cells, or cellular debris in urine. Abnormal cell findings may be important as an indication of urogenital cancer. White or red blood cells, crystals or debris may be important indications of hematuria, a urinary tract infection or blockage.

### BACKGROUND

The National Cancer Institute estimates that 2.3% of people in the U.S. will be diagnosed with bladder cancer at some point over their lifetime and that there are 700,000 people currently living with bladder cancer. There are approximately 74,000 new bladder cases each year. Approximately 50-70% of all bladder cancers recur, of which 33% will progress to myoinvasive disease. Overall, about 18,000 people die from bladder cancer each year. The grim prognosis of non-muscle-invasive bladder cancer progressing to myoinvasive disease highlights the need to survey this high-risk population early on intensely. In addition, follow-up and therapy over a survivor's lifetime make it the most expensive cancer in the United States, with treatment costs ranging from $96,000 to $187,000.

Despite the call from urologists and an ample clinical record showing improved prognosis under early detection, the high-risk population for bladder cancer is not routinely screened for the disease. The search for bladder cancer is typically initiated by finding blood in the urine (Haematuria). Urine cytology combined with cystoscopy is used, but urine cytology typically has a low accuracy (46% sensitivity) and high inter-observer variance. Cystoscopy is a procedure usually performed under light anesthesia, in which a tube is inserted through the urethra to look inside the bladder. Cystoscopy is costly, highly invasive, and shows a big inter-observer and intra-observer variation in the tumor stage and grade interpretation. The sensitivity and specificity of combining cytology and cystoscopy are 85-90% and 65-70% to detect exophytic tumors and carcinoma in situ, respectively. FDA-approved systems for diagnosing and monitoring bladder cancer do not meet sensitivity and specificity requirements (e.g., the NMP22 determination). In contrast, other tests have such high costs that their use in daily health practice is limited. Current studies using molecular liquid biopsy approaches have not yielded a marketable diagnostic. The need for a non-invasive diagnostic to detect and then monitor for bladder cancer with high sensitivity and specificity is still a high priority.

Similarly, non-invasive diagnostics with high sensitivity and specificity are unavailable for other urogenital cancers. Although most are less prevalent than bladder cancer, other urogenital cancers still affect substantial portions of the population, and many have similarly grim prognoses; even those with better prognoses are often not detected until more invasive treatments, such as surgical removal of a kidney, are necessary.

Prostate cancer is another very prevalent type of urogenital cancer that can range from a common form that is so benign clinicians have been increasingly requesting that it not be designated a cancer at all to rapidly metastasizing and typically fatal forms. Currently, the most-used diagnostic for prostate cancer, blood PSA levels, is non-invasive, but, unfortunately, it has several limitations that lead to missed diagnoses, particularly early diagnoses of some aggressive types of prostate cancer, and that also lead to the need for other, more invasive tests to confirm that more benign forms of the disease can be monitored without treatment. A non-invasive prostate cancer test with improvements in the ability to detect early stages of more aggressive types of prostate cancer and/or the ability to differentiate forms of prostate cancer with different risks and requiring different treatments is also still a high priority.

### BRIEF SUMMARY

The present disclosure provides an artificial intelligence (AI)-based urine cell detection method comprising: generating, for a plurality of objects in a urine sample and by the optical tomography system, a 3D image of an object from the urine sample; and evaluating the 3D image using 3D AI-based classification to determine if the object is an abnormal cell or an object other than an abnormal cell.

The present disclosure provided the following additional aspects of the preceding method, which may also be combined with one another in any manner:
prior to generating the 3D image, the method comprises: generating, by an optical tomography system, a 2D image of the object from the urine sample; and evaluating the 2D image using 2D AI-based classification to determine if the object is likely an abnormal cell or likely to be an object other than an abnormal cell;
the method has a pre-selected 2D AI-based classification specificity value for abnormal cells of at least 65%;
the method has a pre-selected 2D AI-based classification sensitivity value for objects other than abnormal cells of at least 90%;
the method has a pre-selected 2D AI-based classification specificity value for objects other than abnormal cells of at least 65%;
the method has a pre-selected 2D AI-based classification sensitivity value for abnormal cells of at least 90%;
the method has a pre-selected 2D AI-based classification specificity value for abnormal cells of at least 65%;
the method has a pre-selected 2D AI-based classification sensitivity value for objects other than abnormal cells of at least 90%;
the method has a pre-selected 2D AI-based classification specificity value for objects other than abnormal cells of at least 65%;
abnormal cell classifications are reflected in an abnormal cell count, which correlates with a patient who provided the urine sample has urogenital cancer, is at a higher than normal risk for having urogenital cancer, is it at an average or lower risk for having urogenital cancer, has a risk of having urogenital cancer above a pre-selected threshold, or has a risk of having urogenital cancer below a pre-selected threshold;
the urogenital cancer is kidney cancer, bladder cancer, urethral cancer, or prostate cancer;
the urine sample comprises abnormal cells, urothelial cells other than abnormal cells, squamous cells, columnar cells, white blood cells, debris, cell clusters, and any combinations thereof;
the method further comprises, prior to generating any image, pre-processing the urine sample to stain the plurality of cells with an agent that facilitates generating the 2D image, if generated, evaluating the 2D image using 2D AI-based classification, if so classified, generating the 3D image, or evaluating the 3D image using 3D AI-based classification;
the method further comprises, prior to generating any image: embedding the urine sample in an optical medium and injecting the optical medium with embedded sample into a capillary tube; and loading the capillary tube into the optical tomography system so that the capillary tube is between an illumination source and objective lens of the optical tomography system;
generating, by the optical tomography system, the 2D image of the cell comprises the optical tomography system sweeping a focal plane of the optical tomography system in 1 μm steps across a single cell to generate a single-plane 2D image of the single cell at each step, compiling a plurality of single-plane 2D images of the single cell, and filtering the set of single-plane 2D images of the single cell to generate a representative 2D image of the cell that is evaluated;
the representative 2D image of the cell is an image of a central portion of the cell;
evaluating the 2D image using 2D AI-based classification comprises determining values for a plurality of 2D image cell feature measurements;
the cell feature measurements comprise object shape features, cell-shape features, cytoplasm features, cell nucleoli features, distribution of chromatin, nuclear-size features, nuclear-texture features, other morphometric elements, or any combination thereof;
evaluating the 3D image using 3D AI-based classification comprises determining values for a plurality of 3D image cell feature measurements;
the cell feature measurements comprise object shape features, cell-shape features, cytoplasm features, cell nucleoli features, distribution of chromatin, nuclear-size features, nuclear-texture features, other morphometric elements, or any combination thereof;
the method further comprises determining if the object is a red blood cell (RBC) and performing all steps as if the RBC were the abnormal cell;
the method further comprises using a total RBC count to determine whether the patient has urogenital cancer, is at a higher than normal risk for having urogenital cancer, is it at an average or lower risk for having urogenital cancer, has a risk of having urogenital cancer above a pre-selected threshold, or has a risk of having urogenital cancer below a pre-selected threshold;
the method further comprises determining if the object is a white blood cell (WBC) or a specific type of WBC and performing all steps as if the WBC or specific type of WBC were the abnormal cell;
the method further comprise using the total WBC or type of WBC count to determine whether the patient has urogenital cancer, is at a higher than normal risk for having urogenital cancer, is it at an average or lower risk for having urogenital cancer, has a risk of having urogenital cancer above a pre-selected threshold, or has a risk of having urogenital cancer below a pre-selected threshold;
the method further comprises using the total WBC count or type of WBC count to determine whether the patient has a urinary tract infection;
the method further comprises using the total WBC count or type of WBC count to determine whether the patient is responding to immune checkpoint inhibitor therapy for urogenital cancer;
the method further comprises determining if the object is a uric acid crystal and performing all steps as if the uric acid crystal were the abnormal cell;
the method further comprises using a total uric acid crystal count to determine whether the patient has urogenital cancer, is at a higher than normal risk for having urogenital cancer, is it at an average or lower risk for having urogenital cancer, has a risk of having urogenital cancer above a pre-selected threshold, or has a risk of having urogenital cancer below a pre-selected threshold;
the method further comprises using a total uric acid crystal count to determine if the patient has a kidney stone;
the method further comprises determining if the object is a cell cluster and performing all steps as if the cell cluster were the abnormal cell;
the method further comprises using a total cell cluster count to determine whether the patient has urogenital cancer, is at a higher than normal risk for having urogenital cancer, is at an average or lower risk for having urogenital cancer, has a risk of having urogenital cancer above a pre-selected threshold, or has a risk of having urogenital cancer below a pre-selected threshold.

The disclosure further provides an object classification system comprising an optical tomography system operable to: generate a 3D image of an object in a urine sample; and evaluate the 3D image using 3D AI-based classification to determine if the object is an abnormal cell or an object other than an abnormal cell.

The present disclosure provided the following additional aspect of the preceding system:
the optical tomography system is further operable to: generate a 2D image of the object; and evaluate the 2D image using 2D AI-based classification to determine if the object is likely an abnormal cell or likely an object other than an abnormal cell.

The present disclosure further provides an optical tomography system operable to perform any of the above methods.

### DESCRIPTION OF THE DRAWINGS

The patent or application file contains at least one drawing executed in color. Copies of this patent or patent application publication with color drawing(s) will be provided by the Office upon request and payment of the necessary fee.

The disclosure may be better understood through reference to the following detailed description in conjunction with the drawings, which are provided as examples only, in which like elements are indicated by letters (e.g., 40a, 40b, 40c), and in which:

FIG. 1 is a schematic representation of an optical tomography system that may be used in the present disclosure;

FIG. 2 is a schematic representation of the optical tomography system as operated to acquire a plurality of 2D images of a urine cell that may be used in the present disclosure;

FIG. 3 is a logic diagram that shows a method for analyzing, comparing and classifying cells from a urine sample;

FIGS. 4A-H are images of urine cells after various preparation treatments;

FIG. 5 is a set of example 2D images of UM-UC-3 bladder cancer cells obtained using an optical tomography system of the present disclosure and identified as abnormal by an AI trained for the detection of lung cancer;

FIG. 6 is a set of 2D images of squamous cells, columnar cells, abnormal cells, normal urothelial cells (normal), debris, or other cells, which include small clusters, from patient urine samples. Other cells are cell clusters, for example, macrophages plus white blood cells.

FIG. 7 is a box plot of positive rates (%) for abnormal cells from patients without bladder cancer, patients without cancer but who previously had bladder cancer, patients with atypical cells exhibiting cytologic atypia (CA), patients with atypical cells with architectural atypia with minimal cytologic atypia (AACA) and patients with malignant cells from high-grade urothelial carcinoma (HGUC); the upper and lower horizontal lines indicate the minimum and maximum values; the top and bottom edges of the boxes indicate the 75th and 25th percentiles for the data; the horizontal line inside the box indicates the median for the data; and the diamond indicates the mean for the data.

FIG. 8 is a plot of the coefficient of variation (μ/σ) of the positive rate vs. the number of urine cells analyzed.

FIG. 9 shows a system diagram that describes an example implementation of a computing system(s) for implementing embodiments described herein.

### DETAILED DESCRIPTION

The present disclosure relates to a urine cell detection method and system that includes an AI-based classification method and system. The method and system may detect abnormal cells among a plurality of urine cells in a urine sample. The urine sample may be obtained from a patient in a conventional manner. Abnormal cells may be cancerous, but they may also include non-cancerous urothelial cells that are not normal. The system and method may use an abnormal cell count or abnormal cell positive rate to determine if the patient has urogenital cancer, if the patient has an increased risk of urogenital cancer, which may be further classified by degree of risk, or if the patient has low or higher than normal risk of urogenital cancer. In particular, the system and method may make the determination based on the count of abnormal cells above a threshold, or the positive rate, in which abnormal cell counts are normalized by total object or other cell counts. Depending on the determination, the patient may be treated for urogenital cancer or monitored more frequently than patients without any increased risk of urogenital cancer.

The threshold for cell counts referenced herein, other than in the context of the number of objects or cells required to be processed to ensure the accuracy of the assay, may refer to a total number of the referenced type of cell(s) or a positive rate derived from the total number of the referenced type of cell(s) divided by the total number of another type of cell(s) or objects.

The term “cancer” refers to a hyperproliferation of cells that results in unregulated growth, lack of differentiation, local tissue invasion, or metastasis.

“Urogenital cancer” as used herein includes any cancer arising in part of the urinary tract, including the kidneys, ureters, bladder, and urethra, and prostate cancer. In some embodiments, the urogenital cancer arises from and/or causes abnormalities in urinary tract epithelial cells (also referred to a “urothelial cells”), which include cells found in the urinary tract, including epithelial cells found in the glomerulus, ureters, bladder, and urethra. Even urogenital cancers that do not arise from urothelial cells, such as prostate cancer, which arises from endothelial cells outside the urinary tract, can cause detectable abnormalities in urothelial cells, particularly nearby urothelial cells, such as urethra urothelial cells near the prostate.

In some embodiments, the tissue-based type of cancer, such as bladder cancer, kidney cancer, urothelial cancer, or prostate cancer, may be specified based on features of abnormal cells. In some embodiments, a more specific type of cancer may be determined, such as the stage of bladder cancer, or whether prostate cancer is a low or high-grade adenocarcinoma, or a more aggressive cancer, such as a small cell carcinoma or squamous cell carcinoma. In some embodiments, a type of atypical urothelial cells may be specified based on features of abnormal cells.

Although many embodiments and examples herein focus on bladder cancer as a model, the systems and methods of this disclosure are applicable to other urogenital cancers because such cancers also result in atypical urothelial cells in the urine.

In some embodiments in which bladder cancer is detected, the bladder cancer may be a carcinoma, such as high grade urothelial carcinoma (HGUC), including carcinoma in situ (CIS), or low grade urothelial cancer (LGUC). Abnormal cells may also include cells exhibiting cytologic atypia (CA), which are likely to progress to HGUC if cancer develops, or cells exhibiting architectural atypia with minimal cytologic atypia (AACA), which are likely to progress to LGUC if cancer develops.

In some embodiments, red blood cells, debris, or cell cluster counts, described in further detail below, may also be used to make further determinations regarding urogenital cancer, such as bladder cancer, or the risk of urogenital cancer, such as bladder cancer. In some embodiments, one or more may be classified as an object other than an abnormal cell.

In some embodiments, white blood cells (WBCs) may also be detected and a white blood cell count determined. If the sample has a white blood cell count above a white blood cell threshold, then the sample is designated as positive for infection, and the patient may be treated accordingly.

In some embodiments, red blood cells (RBCs) may also be detected an a red blood cell count determined. If the sample has a red blood cell count above a red blood cell threshold, then the sample is designated as positive for red blood cells and the patient may be further evaluated and treated appropriately for haematuria. In some embodiments, the system and method may detect the presence of a lower number of red blood cells than conventional cytological methods, allowing for more rapid identification of diseases or disorders or possible complications of urogenital cancer.

In some embodiments, the system and method may also detect cellular debris, which may be indicative of non-cancerous diseases such as a kidney stone, or possible complications of some urogenital cancer. If the cellular debris count exceeds a debris threshold, the sample may be designated as positive for debris.

In some embodiments, the system and method may also detect urothelial cell clusters (also referred to herein as clusters or aggregates), which may indicate bladder cancer because bladder cancer cells tend to form clusters. Urothelial cell clusters, or the absence thereof, may also indicate different urogenital cancers, depending on whether such cancer is associated with cell clusters. If the cell cluster count exceeds a cluster threshold, the sample may be designated as positive for cell clusters.

Several thresholds may be set for white blood cells, red blood cells, debris, or cell clusters to indicate different count levels for these in a urine sample that correlate with different disease states or risks.

The system and method may use tools not available in traditional pathology methods, such as AI-based classifications, enhanced images, image analysis tools, 2D images, 3D images, and image storage and comparisons.

In some embodiments, the optical tomography system may be a CELL-CT® system (VisionGate, Inc., Washington, USA).

In some embodiments, the treatment of urine to enrich a particular type of cell in a sample may be unnecessary. Most cells found in urine may be useful in the present system and method.

### Optical Tomography System

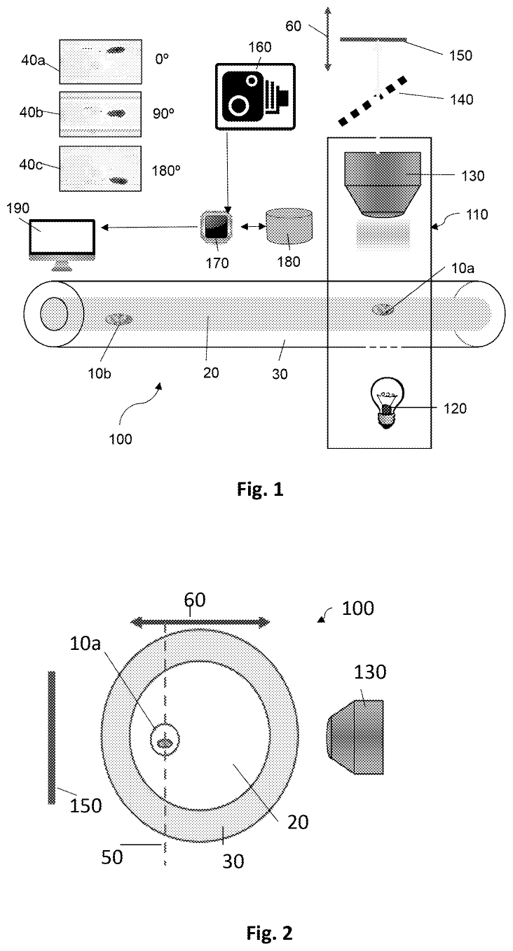

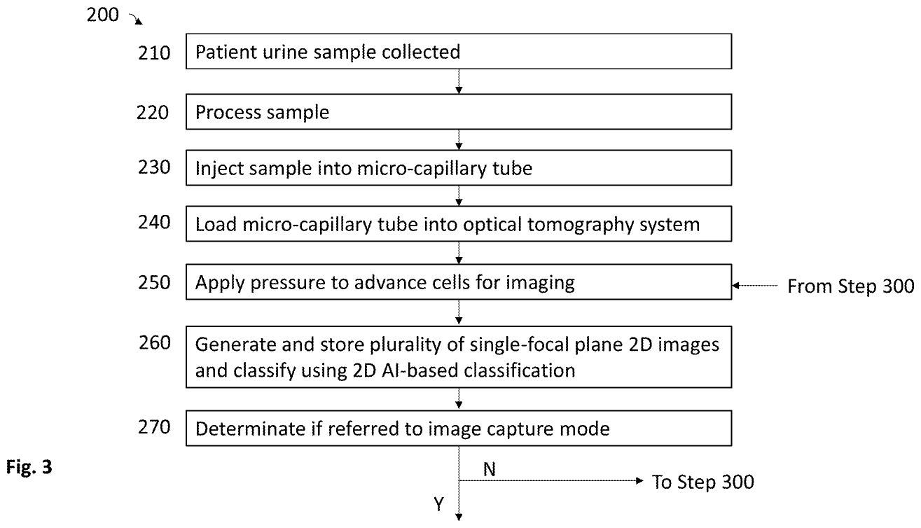

Referring now to FIG. 1 and FIG. 2, a system of the present disclosure may include, or a method of the present disclosure may be carried out using optical tomography system 100, which may be used to produce both 3D images and 2D images of a urine object 10 (such as object 10a or object 10b). Although the operation of the optical tomography system is described for acquiring images of one object 10, in a volume of optical medium in the optical path of a high-magnification microscope, images of multiple objects 10 within the same volume of optical medium may be acquired. Furthermore, images sufficient to classify debris or clusters may also be acquired.

The optical tomography system 100 may include a cell imaging system 110, which includes an illumination source 120 optically coupled to an objective lens 130. In this system, illumination passes through the micro-capillary tube 30 and any intervening object 10 before reaching the objective lens 130.

In embodiments disclosed herein, the optical tomography system 100 may be operated to generate a plurality of 2D images of object 10 in cell search mode. In such embodiments, the objective lens 130 has a focal plane 50 that moves as the objective lens 130 sweeps across the micro-capillary tube 30 and any object 10 in a back-and-forth direction 60 to produce a plurality of 2D images (not shown). This method of generating 2D images by moving the objective lens 130 is different than the method of producing 3D images (described below) using pseudo-projection images that are generated by vibrating the mirror 150. The processor 170 is operable to receive the plurality of 2D images. In some embodiments, a 2D image of the central portion, such as the center of object 10 is designated to be a representative 2D image.

In some embodiments disclosed here, in which the optical tomography system 100 may be operated to generate 3D images of the object 10, the illumination passes through the objective lens 130 to a beam-splitter 140, which causes part of the illumination to be deflected to a mirror 150 and reflected to the beam-splitter 140 before being transmitted to a high-speed camera 160, and another part of the illumination to be transmitted directly through the beam-splitter 140 to the high-speed camera 160, to generate pseudo-projection images 40 of the object 10 contained in an optical medium 20 in a micro-capillary tube 30.

During 3D imaging, also referred to as projection image capture mode, at least one pseudo-projection image 40 of the object 10 is generated by scanning the volume occupied by the object 10 by vibrating mirror 150 in direction 60 (typically using an actuator, such as a piezo-electric motor, not shown), thus sweeping the plane of focus 50 through the object 10 and then integrating the image to create the pseudo-projection image from a single perspective. Additional pseudo-projection images are obtained by rotating the micro-capillary tube 30. The pseudo-projection images are each a single image representing a sampled volume with an extent greater than the depth of field of the objective lens 130. The high-speed camera 160 generates, for each object 10, a plurality of pseudo-projection images 40 that correspond to a plurality of axial micro-capillary tube rotation positions, examples of which are illustrated as 40a, 40b, and 40c in FIG. 1. In some embodiments, 500 pseudoprojection images are generated as the micro-capillary tube 30 is rotated through 360°.

In some embodiments, optical tomography system 100 is communicatively coupled to a processor 170 operable to receive the plurality of pseudo-projection images 40 from the high-speed camera 160 and use the pseudo-projection images 40 to generate a 3D image (not shown) of the object 10.

Images before or after manipulation by the processor 170 may be stored in communicatively coupled memory 180. Patient background data, such as patient identifier data, prior history of cancer, risk factors, and other health data associated with the images and sample, may be stored in communicatively coupled memory 180, as may patient health data.

In some embodiments, the processor 170 is then further operable to perform AI-based classifications of the representative 2D image using 2D cell classifiers as described herein to determine if the object 10 has abnormal features, and, after generating 3D images for a pre-selected number of normal cells, thereafter only generate 3D images of abnormal cells.

The processor 170 may send data regarding the object 10, or data relating to or derived from a plurality of objects 10 contained in a urine sample to a communicatively coupled output 190. Data sent to the output 190 may include 2D or 3D images of one or more objects 10, or a summary of cells in a urine sample analyzed by the optical tomography system 100.

In some embodiments, the cell imaging system 110 includes the illumination source 120, the objective lens 130, the beam-splitter 140, the mirror 150, and the high-speed camera 160.

In some embodiments, the optical tomography system 100 further includes the processor 170, any communicatively coupled memory 180, and the communicatively coupled output 190.

In certain embodiments, the optical tomography system 100 further includes the micro-capillary tube 30, the optical medium 20, or one or more objects 10, but in other embodiments, the optical tomography system 100 does not include one or more of these potential components, although they may be supplied for operation of the system.

### Urine Cell Detection Methods

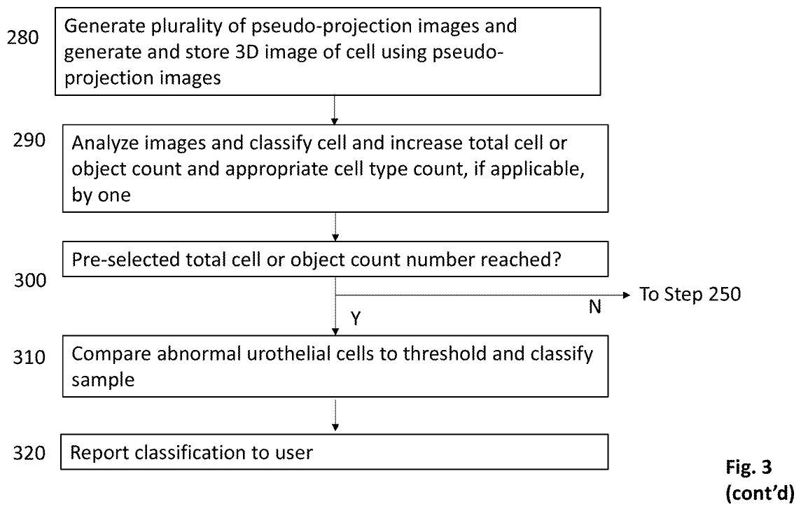

FIG. 3 describes a urine cell detection method 200, which may be performed using an optical tomography system, such as the optical tomography system 100. Elements of the optical tomography system 100 are referenced in this description of the urine cell detection method 200 as examples. Similar components of different optical tomography systems may also be used in connection with the urine cell detection method 200.

The urine cell detection method 200 includes step 210, in which a urine sample is collected from a patient. The sample may be collected in a conventional manner used for urine samples intended for cytological analysis. The urine sample may be stored under any condition that avoids cells from being degraded to the point where limited cellular content remains at the time of optical tomographic analysis. For example, the urine sample may be refrigerated. In some embodiments, the urine sample may be stored for up to 6 hours, 12 hours, 24 hours, or 48 hours after collection before being prepared for optical tomographic analysis. Storage time and conditions, such as the use of refrigeration or storage at room temperature, may be noted, particularly when debris or cluster counts are obtained, as these conditions may affect cell death and resulting debris or cellular aggregation and may, therefore, be relevant to any diagnoses based on these counts.

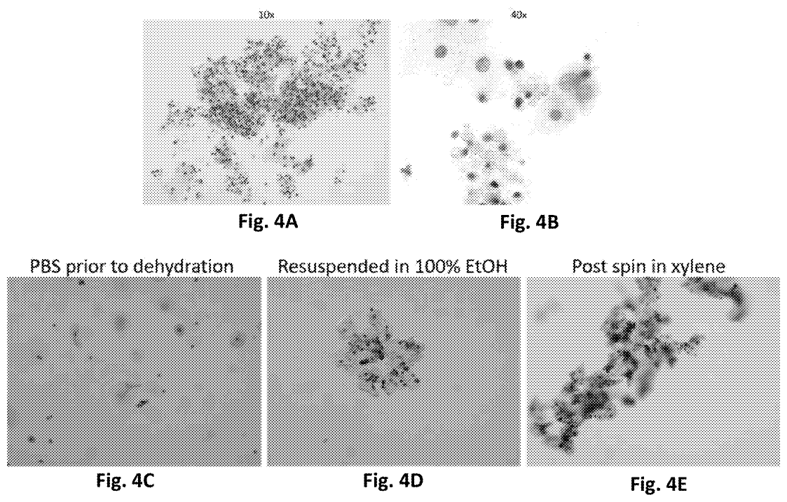

In some embodiments, the urine sample may contain urothelial cells, which may have abnormal features (referred to as “abnormal cells”) or objects other than abnormal cells, such as urothelial cells that do not have abnormal features, squamous cells, such as normal squamous intermediate cells, columnar cells, macrophages, other WBCs, RBCs, debris, clusters, a low number of other cells, such as kidney cells, or other objects not relevant for the identification of bladder cancer. Example images of urine cells that may be found in a urine sample are provided in FIG. 4.

In some embodiments, the urine sample is not enriched for any cell type, such as urothelial cells. Urothelial cells typically represent a large proportion of urine cells in any urine sample, such that enrichment for urothelial cells is not necessary.

In step 220, the urine sample is processed for analysis. For example, the urine sample may be centrifuged to concentrate the plurality of objects 10 prior to placement in optical medium 20.

Processing may optionally include staining to render any of the plurality of objects 10 or any features, such as the nucleus of any of the plurality of objects 10, easier to detect using the optical tomography system 100, or otherwise staining or treating the cells with an agent that facilitates generating the plurality of 2D images of the object 10, evaluating any of the plurality of 2D images, generating a 3D image of the object 10, or analyzing the 3D image. In specific embodiments, the plurality of objects 10 may be stained with a chromatin stain, such as a hematoxylin or bluing reagent. Bluing reagents may also render the nuclear membrane more readily detectable. In some embodiments, only a hematoxylin or bluing reagent is used. In other embodiments, the plurality of objects 10 is stained with both a hematoxylin and a bluing reagent.

The plurality of objects 10 may be treated to ameliorate aggregation. Suitable treatments may include washing the cells, for example in phosphate buffered saline (PBS) prior to staining, washing the cells in distilled water (dH₂O) prior to any ethanol dehydration, or resuspending an ethanol dehydrated cell pellet in xylene. Treatments to ameliorate aggregations may be avoided if cluster count is detected in the overall method and used for any clinical determinations.

Following any optional staining or ameliorations of aggregation, the sample processing 220 includes placing the objects 10 contained in the sample in an optical medium 20.

The optical medium 20 may be any medium reasonably expected to maintain the objects 10 intact during the expected duration of time prior to and during optical tomography. The optical medium 20 may also have a viscosity that allows movement of the optical medium 20 through a micro-capillary tube 30. The optical medium 20 may also not interfere with image generation by the optical tomography system 100. In particular, the optical medium may have an optical index that matches the optical index of other components of the optical tomography system 100 and the micro-capillary tube 30 through which light passes during image acquisition. Typically, the optical tomography system 100 components, through which light passes, and the micro-capillary tube 30 also have a matching index. For optimal optical tomography operation, any changes in light movement should be due to encountering the object to be imaged, not changes in the optical index of other components or objects in the light path.

In step 230, the optical medium 20 containing a plurality of objects 10 from the urine sample is injected into a micro-capillary tube 30. In some embodiments, the micro-capillary tube 30 may have an outer diameter of 500 μm or less, for example, between 30 μm and 500 μm. In some embodiments, the micro-capillary tube 30 may have an inner diameter of 400 μm or less, for example, between 30 μm and 400 μM, such as 60 μm. In one embodiment, the entire portion of the urine sample to be analyzed is placed in one micro-capillary tube 30. In another embodiment, the portion of the urine sample to be analyzed is placed in a plurality of micro-capillary tubes 30, which may be evaluated sequentially. In still another embodiment, the sample may be pumped through the micro-capillary tube 30 from a sample reservoir.

In the step 240, the micro-capillary tube 30 is loaded into the optical tomography system 100 so that the micro-capillary tube 30 is between the illumination source 120, and the objective lens 130.

In step 250, the optical medium 20 and any objects 10 contained within it are advanced into (prior to the initial step 240) or through the micro-capillary tube 30 by applying pressure at one end of the micro-capillary tube, such that a different volume of the optical medium 20 carrying a different portion of the urine sample is in the optical path of the optical tomography system 100, between the illumination source 120 and the objective lens 130. In some embodiments, a plunger (not shown) is used to advance the optical medium 20. For instance, a plunger may be applied to a reservoir (not shown) of the optical medium 20 and urine sample connected to the micro-capillary tube 30, forcing additional optical medium 20 from the reservoir into the micro-capillary tube 30.

Next, in step 260, the optical tomography system generates and stores a plurality of single-focal plane 2D images of the object 10 in cell search mode. In some embodiments, in the cell search mode, the optical tomography system 100 sweeps the focal plane 50 through the object 10 in the direction 60 at 1 μm intervals to capture a series of 2D images. To determine the location of and preserve the image of at least one object 10, at least a portion of the series of single-focal plane 2D images are compiled and filtered by the processor 170 to determine if the 2D images contains features associated with a object 10 or other solid object in the optical medium 20, such as being dark as compared to the optical medium 20. Multiple objects 10 may be identified in the same volume of optical medium 20 in the optical path of the optical tomography system 100.

In some embodiments, 2D AI-based classification may be employed to analyze the plurality of 2D images of the object 10. The AI-based classification method may determine if the object 10 is likely an abnormal cell, likely a urothelial cell other than an abnormal cell, a squamous cell, a columnar cell, a macrophage or other WBC, a RBC, debris, or a cluster.

In step 270, the optical tomography system determines if the object 10 is of a type to be referred to projection image capture mode. If the object 10 is of such a type, the method proceeds to step 280. If object 10 is not of such a type and is excluded from image capture mode, the total object count and any other relevant count is increased by one, and the method moves to step 300.

In some embodiments, debris or clusters may be detected by 2D AI-based classification and excluded from projection image capture mode. If a debris or cluster count is maintained in the method, then the relevant count is increased by one, and the urine cell detection method 200 moves on to step 300.

In some embodiments, any of squamous, columnar, macrophage or other WBC, or RBCs may be detected by 2D AI-based classification and excluded from projection image capture mode. The total object count is increased by one and, if a count of any such cell type is maintained in the method, then the relevant count is also increased by one, and the urine cell detection method 200 moves on to step 300.

In some embodiments, objects other than an abnormal cell may be detected by 2D AI-based classification and excluded from projection image capture mode. The total object count is increased by one and, if a count of such objects is maintained in the method, then that count is also increased by one, and the urine cell detection method 200 moves on to step 300. In some embodiments, a count of only some objects other than an abnormal cell may be maintained, for example, a count of only urothelial cells may be maintained. In such embodiments, the 2D AI-based classification may determine that the cell is a urothelial cell, but not an abnormal cells (such as a urothelial cell having abnormal feature), and increase the count of total urothelial cells by one. In other embodiments, the count of total cells that are not abnormal cells, squamous cells, and columnar cells may be increased by one. In other embodiments, the total number of cells that are not urothelial cells and squamous cells not having abnormal features may be increased by one.

In some embodiments, the sample may be rejected prior to complete analysis based on 2D AI-based classification. For example, RBCs, debris, and clusters are all very readily distinguishable using 2D AI-based classification. If more than a threshold number of objects 10 are classified as an RBC, debris, or cluster by 2D AI-based classification (or by 3D AI-based classification in methods that do to include any 2D classifications), then the urine cell detection method 200 may terminate by providing a message that the sample was rejected.

In some embodiments, only objects 10 identified as likely abnormal cells by 2D AI-based classification may proceed to projection image capture mode. In such embodiments, when a object 10 is not identified using 2D AI-based classification as likely an abnormal cell, then the total object count is increased by one, and the urine cell detection method 200 moves on to step 300.

In other embodiments, objects 10 identified as likely abnormal cells, or, optionally, also squamous, or columnar by 2D AI-based classification may referred to projection image capture mode. In contrast, objects 10 identified as objects other than an abnormal cell are excluded from projection image capture mode.

In some embodiments, a set number of objects 10 classified using the plurality of 2D images as excluded from projection image capture mode are, nevertheless, processed in projection image capture mode and, optionally, also classified using 3D AI-based classification to allow assessment of 2D AI-based classification accuracy or to provide representative images.

In step 280, the method generates a plurality of pseudo-projection images of the object 10 in projection image capture mode and, also generates and stores a 3D image of the object 10 using the pseudo-projection images. In projection image capture mode, the optical tomography system 100 generates a plurality of pseudo-projection images 40 by vibrating the mirror 150.

In step 280, the processor 170 uses at least a portion of the plurality of pseudo-projection images 40 to generate a 3D image of the object 10. Typically, all pseudo-projection images 40 are used to generate the 3D image of the object 10. However, if pseudo-projection images 40 are determined to be of poor quality or likely to contain errors, the 3D imaging of the cell may be discontinued. In addition, typically, pseudo-projection images 40 that cover 360 degrees of rotation around the object 10 are used to generate the 3D image, but pseudo-projection images that cover as little as 180 degrees of rotation around the object 10 may be used.

In step 290, the plurality of 3D images is used in combination with 3D AI-based classification to analyze the object 10 and classify the object 10 as abnormal cells if the object 10 is such a cell. The object 10 may also be classified as urothelial and not having abnormal features, squamous, columnar, macrophage or other WBC, or RBC. The object 10 may also be identified as debris or a cluster rather than a single cell. After 3D AI-based classification as a cell, a total object count is increased by one, if not already increased by one during 2D imaging, or, in instances where 3D imaging reveals the object to be something other than a cell, any cell count from 2D imaging may be decreased by one. If the object 10 is identified by 3D AI-based classification as a abnormal cell, this count is increased by one. If the object 10 is identified by 3D AI-based classification as an object other than an abnormal cell, the relevant count, if maintained, may be increased by one. In some embodiments, specific counts for particular objects, such as urothelial cells that are not abnormal, WBCs, total cells, or total urothelial and squamous cells that are not abnormal, may be maintained and also increased by one if the object is identified by 3D AI-based classification to be the type of object counted.

In both 2D and 3D AI-based classification, in which the processor 170 may detect and measure any of a plurality of cell features in the 2D or 3D images. In some embodiments, the processor 170 detects a nucleus portion and a non-nucleus cellular portion of the object 10 and segments 2D or 3D images into nucleus and non-nucleus portions. In some embodiments, the processor 170 detects and analyzes boundaries of other structures within the cell. Cell features may be in certain categories, including whole-cell, nucleus, cytoplasm, or nucleoli features. Cell features may be of certain types, such as greyscale histogram (e.g. e.t. median, average, 2^{nd}-3^{rd} or -4^{th} statistical moment), spatial distribution (e.g. statistical moments of the Fourier transform), shape (e.g. eccentricity, deviation from spherical ideal), volume, or ratio features (e.g. ratio of nucleus to cytoplasm volume, deviation of the nucleus from cell centroid and center of mass). In some embodiments, the cell feature measurements may include object shape features, cell-shape features, cytoplasm features, cell nucleoli features, distribution of chromatin, nuclear-shape features, nuclear-size features, such as area of nuclear surface, nuclear-texture features, nuclear invaginations, other morphometric elements, such as ratio of nuclear to cytoplasm volume, average grey value, spatial frequencies, grey moments, geometric moments, or any combination thereof.

In some embodiments, the features analyzed may be determined by a human. In other embodiments, AI-based classification may, in addition, or alternatively include analyzing features determined by AI (which may be the same or a different AI as that performing the AI-based classification), or by analyzing other image components, which may also be determined by AI. Features analyzed in this embodiment may be the same as or different from cell features pre-determined by a human as discussed above.

Classification of cells as abnormal cells may have a pre-selected AI-based classification accuracy, such as a pre-selected AI-based classification sensitivity or a pre-selected AI-based classification specificity. In some embodiments, classification of cells as any one urothelial and not having abnormal features, as squamous, columnar, macrophage or other WBC, or RBC or classification as debris or a cluster may also have a pre-selected AI-based classification accuracy. Accuracy for objects other than abnormal cells may be particularly important where such object is used for diagnostic purposes, such as WBCs above a WBC threshold to diagnose infection, or clusters above a cluster threshold to indicate a higher urogenital cancer, such as bladder cancer risk in patients with abnormal cells counts near a threshold.

For any process or assay, the “sensitivity” of the process or assay is defined as the percent of processed or assayed items (such as cells or the sample as a whole) that are actually positive for a property that are also correctly identified as positive by the process or assay. For example, in the context of cell classification, cells are positive for a property if they are identified as such by a cytologist. In the context of the assay as a whole, the sample is actually positive for malignant cells if such cells are identified by a cytologist or are determined to be present by other methods or by progression of the cancer in a high number of patients who are identified as positive for malignant cells. Also, in the context of the assay as a whole, the sample is positive for a higher risk of urogenital cancer, such as bladder cancer, if a cytologist identifies a significant number of abnormal cells or if a high number of patients who are identified as positive for a higher risk of urogenital cancer, such as bladder cancer progress to having such cancer.

Similar significance of sensitivity applies to other determinations that may be made using the urine cell classification method, such as whether the sample is positive for WBCs, RBCs, debris, or clusters.

For any process or assay, the “specificity” of the process or assay is defined as the percent of processed or assayed items (such as cells or the sample as a whole) that are negative for a property that is also correctly identified as negative by the process or assay. Specificity may be determined in a manner similar to sensitivity, but based on absence of urogenital cancer, such as bladder cancer or lack of progression to urogenital cancer, such as bladder cancer in patients with negative results.

In the context of the categorization of urine cells or the overall assay, for each cell type, or for debris or clusters, the correct identification may be determined using a known identification method, such as microscope-based cytology (e.g. review or slides by a pathologist).

In embodiments in which 2D AI-based classification is implemented, sensitivity of 2D classification of cells as abnormal cells tends to be a more significant measure of accuracy than specificity because only cells that are urothelial cells positive for abnormal features are 3D imaged. In a variation in which all urothelial cells are imaged, but not other cell types, the sensitivity of 2D classification of cells as urothelial is a more significant measure of accuracy than specificity because only cells that are urothelial cells are 3D imaged. In either case, a poor sensitivity for 2D AI-based classification can result in cells that have abnormal features not being 3D imaged and not included in the abnormal cells count, potentially resulting in false negatives with respect to either presence of malignant cells (and associated urogenital cancer, such as bladder cancer), or higher than normal risk of urogenital cancer, such as bladder cancer. In contrast, if specificity is poor, then more cells will be 3D imaged than is needed, unnecessarily slowing the urine cell classification somewhat, but not resulting in a significant number of urothelial cells that have abnormal features being missed and false negatives produced.

In some embodiments, the pre-selected 2D AI-based classification sensitivity for abnormal cells, for urothelial cells that are not abnormal cells, for objects other than abnormal cells, or any combination of these may be at least 90%, at least 95%, at least 98%, or in a range of 90% to 100%, 90% to 99.9%, 90% to 99%, 90% to 98%, 90% to 95%, 95% to 100%, 95% to 99.9%, 95% to 98%, 98% to 100%, 98% to 99.9%, or 98% to 99%.

In other embodiments, the pre-selected 2D AI-based classification specificity for abnormal cells, for urothelial cells that are not abnormal cells, for objects other than abnormal cells, or any combination of these may be at least 65%, at least 70%, at least 75%, or in a range of 65% to 100%, 65% to 99%, 65% to 90%, 65% to 80%, 65% to 75%, 65% to 70%, 70% to 100%, 70% to 99%, 70% to 90%, 70% to 80%, 70% to 75%, 75% to 100%, 75% to 99%, 75% to 90%, or 75% to 80%.

The urine cell classification method 200 may also have a pre-selected 2D rejection rate. The 2D rejection rate is the proportion of the total number of enumerated analyzed cells that are identified as not having a property resulting in referral for 3D imaging. In some embodiments, the 2D rejection rate may be 70% or less, 60% or less, 55% or less, 50% or less, or in a range of 0% to 70%, 1% to 70%, 10% to 70%, 25% to 70%, 40% to 70%, 50% to 70%, 55% to 70%, 60% to 70%, 0% to 60%, 1% to 60%, 10% to 60%, 25% to 60%, 40% to 60%, 50% to 60%, 55% to 60%, 0% to 55%, 1% to 55%, 10% to 55%, 25% to 55%, 40% to 55%, 50% to 55%, 0% to 50%, 1% to 50%, 10% to 50%, 25% to 50%, or 40% to 50%.

Accuracy values may be calculated for AI-based 3D classification as well. Specifically, the 3D AI-based classification method may have a 3D abnormal cell sensitivity that is the percent of cells that are or would be determined to have abnormal features using a known identification method, such as cytology (e.g. review by a pathologist), that are identified by AI-based 3D classification as such (or positive for malignant cells or for higher risk of urogenital cancer, such as bladder cancer). When calculating the 3D abnormal cell sensitivity, the number of false negative cells not referred for 3D imaging by the AI-based 2D classification must be subtracted from the known 3D abnormal cell total object count, as the 3D classifier did not have the opportunity to classify such cells as 3D abnormal cell. A pre-selected minimum 3D abnormal cell sensitivity helps avoid false negative test results. Similar calculations may be made for other accuracy parameters of AI-based 3D classification.

In some embodiments, the pre-selected 3D AI-based classification sensitivity for abnormal cells, urothelial cells that are not abnormal cells, for objects other than abnormal cells, or any combination of these may be at least 75%, at least 85%, at least 90%, at least 95%, or in a range of 75% to 100%, 75% to 99%, 75% to 95%, 75% to 90%, 75% to 85%, 85% to 100%, 85% to 99%, 85% to 95%, 85% to 90%, 90% to 100%, 90% to 99%, 90% to 95%, 95% to 100%, or 95% to 99%. Similar sensitivities may be used for other object types, particularly those used for diagnostic purposes. In general, the AI-based classification sensitivity may be set so that, given the prevalence of the cell type in the sample, the product of sensitivity by prevalence yields the pre-selected number (e.g. four) of each type of normal cell for 99%, 99.9%, or 100% of samples.

In some embodiments, the pre-selected 3D AI-based classification specificity for abnormal cells, urothelial cells that are not abnormal cells, for objects other than abnormal cells, or any combination of these may be at least at least 60%, at least 65%, at least 70%, at least 95%, or in a range of 60% to 100%, 60% to 90%, 60% to 80%, 60% to 70%, 65% to 100%, 65% to 90%, 65% to 80%, 65% to 70%, 70% to 100%, 70% to 90%, 70% to 80% 95% to 100%, of 95% to 99%. Similar sensitivities may be used for other object types, particularly those used for diagnostic purposes. In general, the urothelial cell or abnormal cell AI-based classification specificity may be high, such that, for example, a squamous cell is rarely identified as a urothelial cell.

The urine cell classification method 200 also has pre-selected positive for malignant cell accuracy parameters for the method as a whole. The assay as a whole may also have pre-selected urogenital cancer, such as bladder cancer or risk level accuracy parameters. Urogenital cancer sensitivity, such as bladder cancer sensitivity, is the percentage of patients who actually have urogenital cancer, such as bladder cancer, who are identified as having urogenital cancer, such as bladder cancer, by the urine cell detection method (a positive test result). The risk level accuracy is the percent of patients who have that risk level based on cytological analysis of the sample.

The urine cell classification method 200 may also have an abnormal cell sensitivity is the percent of urothelial cells that are or would be identified as having abnormal features using known identification methods that are identified as abnormal cells by the urine cell classification method 200. The assay's abnormal cell sensitivity may be approximated by multiplying the 2D abnormal cell sensitivity by the 3D abnormal cell sensitivity. The assay abnormal cell specificity is the percent of cells that are or would be identified as not having abnormal features using known identification methods identified as not having abnormal features by the method 200. The assay abnormal cell sensitivity may be approximated by multiplying the 2D abnormal cell specificity by the 3D abnormal cell specificity. In particular, in some embodiments overall specificity=100% (1−FPr_3D×FPr_2D) (FPR=False Positive rate, 3D refers to 3D identification, 2D refers to 2D identification). The assay as a whole may have sensitivity and specificity similar to those recited above for AI-based 3D classification.

In some embodiments in which the assay as a whole also provides another diagnostic determination, such as whether there is blood in the urine sample or whether the patient has an infection, the assay as a whole may have pre-selected accuracy parameters for these diagnostic determinations as well. Accuracy may be assessed against conventional methods or other ways of independently verifying the diagnostic status of the patient.

In step 300, the total object count and any applicable cell type, debris, or cluster counts are compared to pre-selected numbers to determine if the pre-selected number have been reached. If all pre-selected numbers have been reached, the method proceeds to step 310. If not, then the method returns to step 250 to acquire images of additional cells.

In general, the accuracy of any urine cell classification method that uses 3D imaging of the type used in method 200 depends on the number of cells (particularly urothelial cells) that are imaged. A pre-selected threshold number for a particular cell count maybe set to ensure a pre-selected level of accuracy. If this pre-selected threshold number cannot be met before the sample is exhausted, an error message may be generated.

In some embodiments, the pre-selected threshold number may simply be applied to the total object count. In the embodiment described in FIG. 3, such a threshold is used. However, in many embodiments, the pre-selected threshold number may be of any cell type or combination of cells. In a specific embodiment, it may be a pre-selected threshold number of total urothelial cells. In another specific embodiment, it may be a pre-selected threshold number of objects other than an abnormal cell, such as urothelial cells that are not abnormal cells, or non-clustered cells that are not abnormal cells. In yet another specific embodiment that is particularly likely to yield accurate results, it may be a pre-selected threshold number of the total of cells that are not abnormal cells, squamous cells, and columnar cells or a pre-selected threshold number of the total number of urothelial cells and squamous cells not having abnormal features. The pre-selected total object count or total urothelial cell count may, therefore, be at least 250, at least 400, at least 500 at least 600, at least 700, at least 800, at least 900, at least 1000, at least 1100, at least 1200, at least 1300, at least 1400, at least 1500, at least 2000, at least 2500, at least 3000, at least 3500, at least 4000, in a range of between any of 250, 400, 500, 600, 700, 800, 900, 1000, 1100, 1200, 1300, 1400, 1500, 2000, 2500, 3000, or 3500 and any of 400, 500, 600, 700, 800, 900, 1000, 1100, 1200, 1300, 1400, 1500, 2000, 2500, 3000, 3500, or 4000.

In some embodiments, more than one cell is located in a given volume of the optical medium, such that an additional cell may be identified, imaged, and used to update cell counts before the method proceeding to step 310 or returning to step 250.

Step 310 compares the abnormal cell count to at least one threshold number. If the number exceeds the threshold, then the urine sample is classified as positive for malignant cells, which means the patient has urogenital cancer, such as bladder cancer. This comparison may be performed by processor 170.

In some embodiments, if the abnormal cell count is below the threshold for positive for malignant cells, but still higher than an elevated risk threshold, then the urine sample is classified as from a patient at higher than normal risk for developing urogenital cancer, such as bladder cancer.

In some embodiments, gradations of risk may also be implemented in the classification, using additional thresholds. For example, the patient may be classified as at high risk or at low risk for developing urogenital cancer, such as bladder cancer.

In another example, the patient may be classified as having either malignant or pre-cancerous urothelial cells, which may be based on comparisons to abnormal cell thresholds, or based on comparisons of abnormal cells classified as malignant or pre-cancerous to specific thresholds for these groups. For example, the abnormal cell count may be compared to classifiers or thresholds to produce separate cell counts for malignant abnormal cells and pre-cancerous abnormal cells.

In other embodiments, thresholds other than for urothelial cells may also be used to determine the risk of developing urogenital cancer, such as bladder cancer. For example, the patient may have an abnormal cell count above a risk threshold, but below a positive for malignant cells threshold. Such a patient also have a cluster count above a positive for clusters threshold. This combined data from two different types of objects, abnormal cells and clusters, may indicate that the patient is actually at high risk for developing urogenital cancer, such as bladder cancer.

The patient may also have other cell types compared to thresholds for other diagnostic purposes, such as infection, blood in urine, or the presence or likelihood of a condition associated with debris in urine.

In particular embodiments, the methods and systems described herein for use in classifying objects as abnormal cells or objects other than abnormal cells may be used to classify objects as RBCs or objects other than RBCs. Based on a comparison of total RBC count to a pre-selected threshold total RBC count, the urine sample may be classified as positive for RBCs, negative for RBCs, or having an intermediate level of RBCs. Sensitivities and specificities for RBCs may be similar to those described herein for abnormal cells.

RBCs in urine may indicate hematuria, which can be associated with urogenital cancer. Accordingly, information about RBCs in the urine sample may further alert clinicians to the possibility of urogenital cancer in the patient. In some embodiments, the method may also include a further calculation that take into account both the number of abnormal cells an the number of RBCs in the urine sample to indicate the patient's urogenital cancer status.

In other embodiments, the methods and systems described herein for use in classifying objects as abnormal cells or objects other than abnormal cells may be used to classify objects as WBCs or objects other than WBCs. Based on comparison of total WBC count to a pre-selected threshold total WBC count, the urine sample may be classified as positive for WBCs, negative for WBCs, or having an intermediate level of WBCs. Sensitivities and specificities for WBCs may be similar to those described herein for abnormal cells.

WBCs in urine may indicate an infection, which may account for some symptoms that would otherwise be associated with urogenital cancer and may alert clinicians to, for example, the need to treat the infection prior to or concurrently with additional assessments for urogenital cancer. In some embodiments, the type of WBC may also be detected and reported, which may further assist the clinician in determining if the patient likely has an infection or an increased WBC count as an effect of an immune response to cancer (such as an increased immune response seen after administration of immune checkpoint inhibitors). In some embodiments, the method may also include a further calculation that takes into account both the number of abnormal cells and the number of WBCs, and, optionally, the counts by WBC type, in the urine sample to indicate the patient's infection status, urogenital cancer status, response to checkpoint inhibitor therapy status, or any combinations of these.

In other embodiments, the methods and systems described herein for classifying objects as abnormal cells or objects other than abnormal cells may be used to classify objects as uric acid crystals or objects other than uric acid crystals. Based on comparison of total uric acid crystal count to a pre-selected threshold total uric acid crystal count, the urine sample may be classified as positive for uric acid crystals, negative for uric acid crystals, or having an intermediate level of uric acid crystals. Sensitivities and specificities for uric crystals may be similar to those described herein for abnormal cells.

Uric acid crystals in urine may indicate kidney stones, often before the kidney stones are readily detectable by other methods. Kidney stones may account for some symptoms that would otherwise be associated with urogenital cancer and may alert clinicians to, for example, the need to allow the kidney stone to pass prior to or concurrently with additional assessments for urogenital cancer. In some embodiments, in which the patient likely also has a urogenital cancer, treatment of the kidney stones may be altered based on risks posed by the co-occurring cancer or its treatment. In some embodiments, the method may also include a further calculation that take into account both the number of abnormal cells an the number of uric acid crystals in the urine sample to indicate the patient's kidney stone status, urogenital cancer status, or both.

In other embodiments, the methods and systems described herein for use in classifying objects as abnormal cells or objects other than abnormal cells may be used to classify objects as cell clusters or objects other than cell clusters. Based on comparison of total cell cluster count to a pre-selected threshold total cell cluster, the urine sample may be classified as positive for cell clusters, negative for cell clusters, or having an intermediate level of cell clusters. Sensitivities and specificities for cell clusters may be similar to those described herein for abnormal cells.

Abnormal cells tend to form cell clusters more readily than other cells. Accordingly, the presence or amount of cell clusters may be a useful adjunct to determining the patient's urogenital cancer status. In some embodiments, the method may also include a further calculation that take into account both the number of abnormal cells and the number of cell clusters in the urine sample to indicate the patient's urogenital cancer status.

In some embodiments, any combinations of RBCs, WBC, uric acid crystals, and cell clusters may be taken into account, along with the number of abnormal cells in the urine sample, to indicate the patient's urogenital cancer status.

In some embodiments, cell classifications may be reviewed by cytologists who may use the 3D images of an object 10.

Urine cell classifications may also include predicted accuracy data, such as predicted specificity, sensitivity, or other measures of accuracy discussed herein.

In some embodiments, patient data aids cell interpretation by the AI-based classification system. In this manner, data may include one or more of the following: patient age, patient gender, patient prior history with cancer, and patient prior history with non-cancer diseases.

In step 320, the urine cell classification results are provided to the user, for example using output 190, and may be stored, for example in memory 180.

Meyer, M. G., et al. (2015), The Cell-CT® 3-dimensional cell imaging technology platform enables the detection of lung cancer using the noninvasive LuCED sputum test. Cancer Cytopathology, 123: 512-523 (doi.org/10.1002/cncy.21576); Wilbur, D. C., et al. (2015), Automated 3-dimensional morphologic analysis of sputum specimens for lung cancer detection: Performance characteristics support use in lung cancer screening. Cancer Cytopathology, 123: 548-556 (doi.org/10.1002/cncy.21565); U.S. Pat. Nos. 6,519,355, 6,522,775, 6,591,003, 6,636,623, 6,697,508, 7,197,355, 7,494,809, 7,569,789, 7,738,945, 7,811,825, 7,835,561, 7,867,778, 7,787,112, 7,907,765, 7,933,010, 8,090,183, 8,155,420, 8,947,510, 9,594,072, 10,753,857, 11,069,054, and US20200018704, are each incorporated by reference herein in its entirety and specifically as it relates to the components, basic operation, including image formation, including formation of pseudo-projection images, and 3D classifiers of optical tomography systems and cancer detection methods and systems described herein.

In some embodiments, when a urine sample is designated as positive for malignant cells, the patient may be referred for further urogenital cancer, such as bladder cancer testing or they may begin treatment for urogenital cancer, such as bladder cancer. When a urine sample is designated as having cells correlating with a higher than normal risk of urogenital cancer, such as bladder cancer, the patient may be retested at set intervals or otherwise monitored or referred for further testing. When a urine sample is designated as not correlating with any increased risk of urogenital cancer, such as bladder cancer, the patient may be monitored in the same manner as a normal, healthy adult.

In some embodiments, when a urine sample is designated positive for WBCs, the infection may be treated.

In some embodiments, when a urine sample is designated positive for RBCs, the patient may be referred for further testing to determine the cause of blood in their urine (haematuria), if no other cause is apparent.

In some embodiments, when a urine sample is designated positive for debris, the patient may be referred for further testing for a non-cancerous disease or disorder, or the positive result may confirm the presence of a non-cancerous disease or disorder for which the patient has other symptoms.

### Training Methods

The present disclosure further includes a method training AI-based components used in AI-based 2D or 3D cell classification. Classification in both 2D and 3D may be governed by a binary ground truth for the property of interest. For example, the cell may either have abnormal features or not have abnormal features. As another example, the cell may either be a urothelial cell or not be a urothelial cell.

Trained AI-based classification systems and methods may be most accurate when implemented on the same or a very similar type of optical tomography system as used in training. Cells for training purposes have a known type, such as a binary type or a patient has a known disease or risk state, which may also be expressed in a binary fashion. During training, a process computes a plurality of cell feature measurements for each of a plurality of known cells and identifies each cell in a binary fashion. The process of computing the identity from the known type includes many variants such as: Logistic regression, Adaptively boosted logistic regression, Random Forest, Decision trees, Neural networks, such as a convolutional neural network (CNN), or any combination thereof. Adaptively Boosted Logistic Regression uses logistic regression in an iterative loop to improve overall classification accuracy. The identification is compared to the known value. The accuracy of identification is assessed and, if it does not meet pre-set values, an investigation is launched to determine the reason. Performance limitations could be from many sources, for example: Inaccuracies in identification of cell known type, inaccuracies in the method of computation of features, lack of diversity in feature type definition, classifier methods that are not well matched to the feature data, or insufficient number of cells in the training set. This training process may be implemented on either the 2D or 3D AI-based classification method, or both.

Throughout the specification, claims, and drawings, the following terms take the meaning explicitly associated herein, unless the context clearly dictates otherwise. The term “herein” refers to the specification, claims, and drawings associated with the current application. The phrases “in one embodiment,” “in another embodiment,” “in various embodiments,” “in some embodiments,” “in other embodiments,” and other variations thereof refer to one or more features, structures, functions, limitations, or characteristics of the present disclosure, and are not limited to the same or different embodiments unless the context clearly dictates otherwise.

In the present description, any concentration range, percentage range, ratio range, or integer range is to be understood to include any values or subranges within the recited range unless otherwise indicated. It should also be noted that the term “or” is generally employed in its sense including “or” (i.e., to mean either one, both, or any combination thereof of the alternatives) unless the content dictates otherwise. Also, as used in this specification and the appended claims, the singular forms “a,” “an,” and “the” include plural referents unless the content dictates otherwise. The terms “include,” and “have” and their variants are used synonymously and are to be construed as non-limiting. The term “a combination thereof” as used herein refers to all possible combinations of the listed items preceding the term. For example, “A, B, C, or a combination thereof” is intended to refer to any one of: A, B, C, AB, AC, BC, or ABC. Similarly, the term “combinations thereof” as used herein refers to all possible combinations of the listed items preceding the term. For instance, “A, B, C, and combinations thereof” is intended to refer to all of: A, B, C, AB, AC, BC, and ABC.

### EXAMPLES

A lung cancer detection method implemented using a CELL-CT® optical tomography system has been developed that can detect lung cancer at a pre-invasive stage using AI-based classifications of cells. The lung cancer AI was trained using sputum samples from patients with lung cancer and normal patients. Although the morphology of abnormal cells associated with lung cancer and of abnormal cells associated with bladder cancer is not identical, there are similarities. Accordingly, if a CELL-CT® optical tomography system could be combined with AI-based classification of urine cells to detect bladder cancer, such a system trained to detect lung cancer would be expected to also be able to detect bladder cancer, although with less accuracy than a system trained using urine samples from patients with bladder cancer and from normal patients.

The following abbreviations are used in these examples:
Atypical-CA: Exhibiting cytological atypia (CA).
Atypical-ACAA: Exhibiting architectural atypia with minimal cytologic atypia (AACA).
PFMC-HGUC: Positive for malignant cells from high grade urothelial cancer (HGUC).
CIS: Exhibiting carcinoma in situ.
HG-TCC: Exhibiting high grade transitional cell carcinoma (may develop in kidney or bladder)
LG-TCC: Exhibiting low grade transitional cell carcinoma
RCC, Papillary type: Exhibiting papillary renal cell carcinoma.

Cell line UM-UC-3, a malignant urothelial cell line that was isolated from the bladder of a patient with bladder cancer, was used in these examples.

Urine specimens used in these examples were from patients who did not have bladder cancer or cellular abnormalities, or who had abnormalities or malignant cells as determined by cytological analysis or development of cancer detected by other means, including when cytological analysis was erroneously negative. Patient information is provided in Table 1. “Diagnosis” refers to cytological diagnosis. “Type of Cancer” refers to the type of cancer the patient had at the time of sample collection.

TABLE 1
Patient Data

| | | | | Prior | |
|---|---|---|---|---|---|
| | | | | Urothelial | |
| ID | Age | Sex | Diagnosis | Cancer | Type of Cancer |
| N17-5933 | 57 | F | Negative | N | |
| N17-5971 | 68 | M | Negative | Y | Multifocal CIS |
| | | | | | Bladder |
| N17-6000 | 73 | F | Negative | Y | HG TCC kidney |
| N17-6001 | 84 | F | Negative | N | |
| N17-6009 | 87 | F | Negative | N | |
| N17-6039 | 43 | F | Negative | N | |
| N17-6066 | 43 | F | Negative | N | |
| N17-6068 | 79 | M | Negative | Y | LG TCC Bladder |
| N17-6070 | 37 | F | Negative | Y | LG TCC Bladder |
| N17-5884 | 89 | M | Atypical- | Y | HG TCC Bladder |
| | | | CA | | |
| N17-5885 | 74 | M | Atypical - | Y | HG TCC Bladder |
| | | | CA | | |
| N17-5937 | 62 | F | Atypical- | N | RCC, Papillary |
| | | | AACA | | type |
| N17-5942 | 51 | F | Atypical | Y | CIS Bladder |
| | | | AACA | | |
| N17-5949 | 81 | M | Atypical- | Y | HG TCC Bladder |
| | | | AACA | | |
| N17-5989 | 90 | M | Atypical- | Y | HG TCC Bladder |
| | | | AACA | | |
| N17-6071 | 84 | M | Atypical- | Y | LG TCC Bladder |
| | | | AACA | | |
| N17-5941 | 91 | M | PFMC- | Y | HG TCC Bladder |
| | | | HGUC | | |
| N17-6044 | 84 | F | PFMC- | Y | HG TCC Kidney |
| | | | HGUC | | |

Urine specimens were naturally voided, then processed by centrifugation followed by resuspension in PreservCyt fixative. Specimens used were remainders of samples that had saturated cytospin membranes and thus were of high cellular content. Modifications of processing methods in this example may be made with urine samples or urine samples following simple centrifugation, which will have lower cellular content.

### Example 1

### Urine Processing Protocol

Urine specimens were processed and stained without dithiothreitol treatment or cellular enrichment for urothelial cells, then embedded in optical medium. Unlike sputum, urine contains a high proportion of epithelial cells, making an enrichment step unnecessary. Also unlike sputum, urine has little to no mucus, making treatment with dithiothreitol to dissolve mucus unnecessary. Various protocols were tested to evaluate any effects on sample processing

### Staining Protocol A

1. Briefly vortex specimen cup and filter sample through 100μ cell strainer into a 50 mL tube.
2. Transfer filtrate to a 15 mL tube.
3. Centrifuge 500×g for 5 minutes.
4. Pour off supernatant into original specimen cup leaving ~400 μL of residual liquid in tube.
5. Briefly vortex to resuspend pellet and transfer to microfuge tube.
6. Rinse 15 mL tube with 200 μL of saved supernatant from Step 4 and combine into microfuge tube from Step 5.
7. Centrifuge 500×g for 3 minutes.
8. Aspirate supernatant and resuspend pellet in 200 μL 50% ethanol.
9. Centrifuge 500×g for 3 minutes.
10. Aspirate supernatant and resuspend pellet in 200 μL Gill's hematoxylin.
11. Centrifuge 500×g for 3 minutes.
12. Aspirate supernatant and resuspend pellet in 200 μL dH2O.
13. Centrifuge 500×g for 3 minutes.
14. Aspirate supernatant and resuspend pellet in 200 μL Bluing reagent.
15. Centrifuge 500×g for 3 minutes.
16. Aspirate supernatant and resuspend pellet in 200 μL PBS.
17. Centrifuge 500×g for 3 minutes.
18. Aspirate supernatant and resuspend pellet in 500 μL PBS.
19. Filter sample using 35μ cell strainer tube.
20. Remove a 10 UL aliquot for cell count determination using a hemocytometer.
21. Embed cells at ~3,000/μL in optical media.

### Staining Protocol B—For Cell Lines

1. Transfer cells to a 1.5 mL microfuge tube.
2. Centrifuge 500×g for 5 minutes.
3. Aspirate supernatant and resuspend pellet in 200 μL 50% ethanol.
4. Centrifuge 500×g for 3 minutes.
5. Aspirate supernatant and resuspend pellet in 150 μL dH2O and add 50 μL Gill's hematoxylin. Vortex briefly.
6. Centrifuge 500×g for 3 minutes.
7. Aspirate supernatant and resuspend pellet in 200 μL dH2O.
8. Centrifuge 500×g for 3 minutes.
9. Aspirate supernatant and resuspend pellet in 200 μL Bluing reagent.
10. Centrifuge 500×g for 3 minutes.
11. Aspirate supernatant and resuspend pellet in 200 μL PBS.
12. Remove a 10 μL aliquot for cell count determination using a hemocytometer.
13. Embed cells at ~3,000/μL in optical media.
   Staining Protocol C—Similar to A but with No Initial Filtration and Addition of PBS Prior to Gill's and Bluing
1. Briefly vortex specimen cup and distribute to 15 mL tubes.
2. Centrifuge 500×g for 5 minutes.
3. Pour off supernatant into original specimen cup leaving ~400 μL of residual liquid in tube.
4. Briefly vortex to resuspend pellet and transfer to microfuge tube.
5. Rinse 15 mL tube with 200 μL of saved supernatant from Step 4 and combine into microfuge tube from Step 5.
6. Centrifuge 500×g for 3 minutes.
7. Aspirate supernatant and resuspend pellet in 200 μL 50% ethanol.
8. Centrifuge 500×g for 3 minutes.
9. Aspirate supernatant and resuspend pellet in 200 μL Gill's hematoxylin.
10. Centrifuge 500×g for 3 minutes.
11. Aspirate supernatant and resuspend pellet in 200 μL dH2O.
12. Centrifuge 500×g for 3 minutes.
13. Aspirate supernatant and resuspend pellet in 200 μL PBS.
14. Centrifuge 500×g for 3 minutes.
15. Aspirate supernatant and resuspend pellet in 200 μL Bluing reagent.
16. Centrifuge 500×g for 3 minutes.
17. Aspirate supernatant and resuspend pellet in 200 μL PBS.
18. Centrifuge 500×g for 3 minutes.
19. Aspirate supernatant and resuspend pellet in 500 μL PBS.
20. Filter sample using 35μ cell strainer tube.
21. Remove a 10 μL aliquot for cell count determination using a hemocytometer.
22. Embed cells at ~3,000/μL in optical media.
   Staining Protocol D—Same as C Except for Addition of dH2O Wash Prior to Dehydration.
1. Briefly vortex specimen cup and distribute to 15 mL tubes.
2. Centrifuge 500×g for 5 minutes.
3. Pour off supernatant into original specimen cup leaving ~400 μL of residual liquid in tube.
4. Briefly vortex to resuspend pellet and transfer to microfuge tube.
5. Rinse 15 mL tube with 200 μL of saved supernatant from Step 4 and combine into microfuge tube from Step 4.
6. Centrifuge 500×g for 3 minutes.
7. Aspirate supernatant and resuspend pellet in 200 μL 50% ethanol.
8. Centrifuge 500×g for 3 minutes.
9. Aspirate supernatant and resuspend pellet in 200 μL PBS.
10. Centrifuge 500×g for 3 minutes.
11. Aspirate supernatant and resuspend pellet in 200 μL Gill's hematoxylin.
12. Centrifuge 500×g for 3 minutes.
13. Aspirate supernatant and resuspend pellet in 200 μL dH2O.
14. Centrifuge 500×g for 3 minutes.
15. Aspirate supernatant and resuspend pellet in 200 μL PBS.
16. Centrifuge 500×g for 3 minutes.
17. Aspirate supernatant and resuspend pellet in 200 μL Bluing reagent.
18. Centrifuge 500×g for 3 minutes.
19. Aspirate supernatant and resuspend pellet in 200 μL PBS.
20. Centrifuge 500×g for 3 minutes.
21. Aspirate supernatant and resuspend pellet in 500 μL PBS.
22. Filter sample using 35μ cell strainer tube.
23. Rinse filter with 200 μL PBS.
24. Remove a 10 μL aliquot for cell count determination using a hemocytometer.
25. Transfer to microfuge tube.
26. Centrifuge 500×g for 3 minutes.
27. Aspirate supernatant and resuspend pellet in 200 μL dH2O.
28. Embed cells at ~3,000/μL in optical media.

Cell aggregation occurred during the processing of some specimens. Aggregation occurred either during staining using Protocol A (FIG. 4A and FIG. 4B). A large proportion of the sample remained on top of the 35 m cell strainer, as may be seen in the figures. Aggregation also occurred during embedding using Protocol D, and is shown when cells were in PBS prior to ethanol dehydration (FIG. 4C), after cells were resuspended in 100% ethanol (FIG. 4D), and after centrifugation in xylene (FIG. 4E).

Further testing of the staining protocols was conducted using the Jurkat immortalized human T cell lymphocyte cell line. Jurkat cells processed using Protocol A exhibited aggregation. Jurkat cells processed using Protocol D with additional PBS wash steps prior to adding Gill's hematoxylin or bluing reagent were mostly single cells. However, as demonstrated by FIG. 4C, FIG. 4D, and FIG. 4E even Protocol D resulted in some aggregation of urothelial cells during the dehydration and embedding process. Further tests revealed that washing the cells with distilled water prior to ethanol dehydration reduced aggregation (FIG. 4F) and this reduction was maintained when the cells were suspended in xylene prior to centrifugation (FIG. 4G), although some aggregation was still observed while the cells were suspended in xylene after centrifugation (FIG. 4H). If aggregation, such as that shown in FIG. 4H is observed and unprocessed urine sample is available, aggregation may be further mitigated by resuspending the cell pellet after the 100% ethanol wash in 15 μL of xylene and proceeding directly to embedding without centrifugation in xylene.

Based on these experiments, Staining Protocol D is the recommended urine processing protocol.

The following specific reagents and materials were used in this example:
Bluing reagent: Fisher Bluing Reagent 220-106 (Thermo Fisher, US).
Bovine serum albumin: 2% in 1×PBS
Anti-Cytokeratin-FITC CK3-6H5 antibody: Miltenyi, 130-080-101 (Miltenyi, Germany)
Pan-Cytokeratin (C11) Mab, Mouse antihuman: Alexa Fluor® 488 conjugate, Cell Signaling Technology 4523 (Cell Signaling Technology, US) 100 μL used.
Hematoxylin: Gill's #1 hematoxylin (Electron Microscopy Sciences, US)
Optical medium: Nyogel® optical coupling fluid, Nye OCF-452H, index of refraction 1.51 at 589.3 nm (Nye Lubricants, Inc., US)
Fixative: PreservCyt (Hologic, Inc., US)

### Example 2

### Analysis of Urine Samples Using Lung Cancer-Trained AI

2D and 3D images of cells in urine samples processed according to the above Protocol B were collected using the CELL-CT® optical tomography system and classified as abnormal cells, normal urothelial cells (urothelial cells not having abnormal features), squamous cells, columnar cells, or other cells or debris using AI-based classification trained to detect abnormalities in bronchial epithelial cells.

2D images of cells were used to determine if cells were non-target cells, although 3D images were also captured for all cells in this example to allow evaluation of the method.

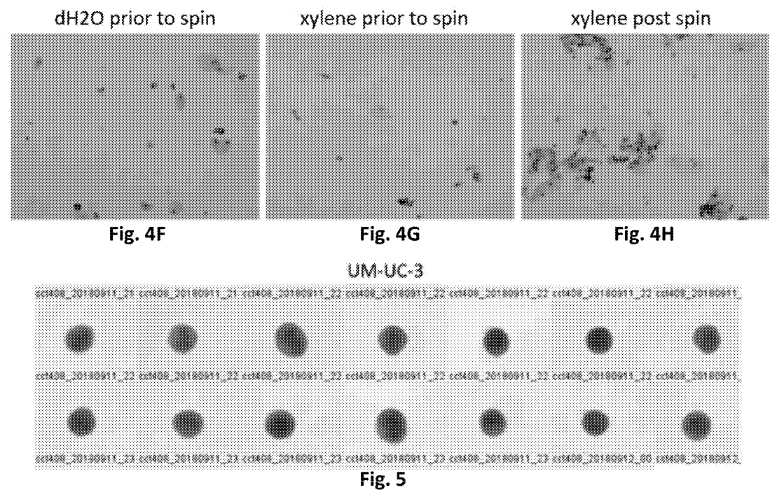

19% of cells in the UM-UC-3 sample were classified as abnormal. Examples of 2D images of cells classified as abnormal are shown in FIG. 5. This demonstrates the ability to identify some abnormal cells using an optical tomography system and trained AI-based classification, with more accurate results likely if the AI were trained using bladder cancer cells.

Patient urine samples as described above were treated as indicated in Table 2 and then analyzed. No aggregation was observed in samples where Protocol D was used.

TABLE 2
Treatment Protocols and Yields for Patient Samples

| | Sample | Staining | Pellet |
|---|---|---|---|
| ID | Volume (mL) | Protocol | Volume (μL) |
| N17-5933 | 15 | A | 60 |
| N17-5971 | 18 | A | 25 |
| N17-6000 | 13 | A | 50 |
| N17-6001 | 12 | D | 30 |
| N17-6009 | 15 | D | 0 |
| N17-6039 | 18 | D | 40 |
| N17-6066 | 4 | D | 5 |
| N17-6068 | 1 | D | 1 |
| N17-6070 | 14 | D | 20 |
| N17-5884 | 15 | A | 25 |
| N17-5885 | 10 | A | 25 |
| N17-5937 | 10 | A | 50 |
| N17-5942 | 16 | A | 40 |
| N17-5949 | 2 | D | 5 |
| N17-5989 | 10 | D | 10 |
| N17-6071 | 10 | D | 10 |
| N17-5941 | 14 | D | 25 |
| N17-6044 | 13 | D | 40 |

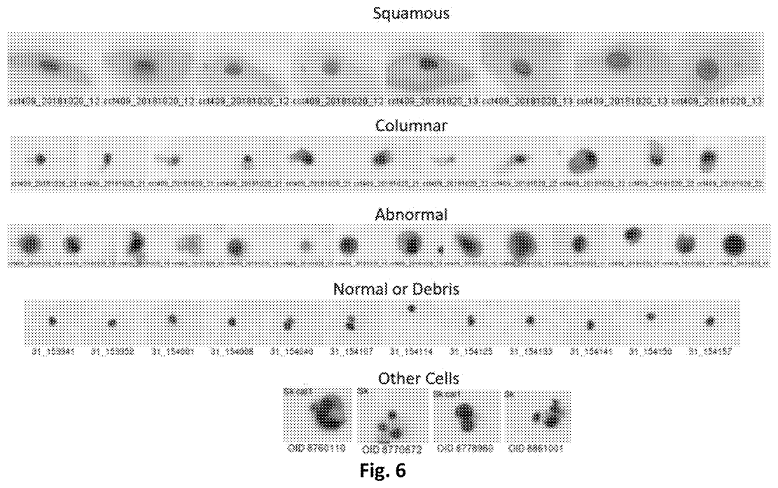

Samples were loaded into disposable specimen containers and analyzed using a version 4 (V4) CELL-CT® optical tomography system. Images were acquired for 275,329 cells in 17 specimens (one specimen, as noted in Table 2, did not yield a usable pellet). FIG. 6 shows representative squamous cells, columnar cells, abnormal cells, normal urothelial cells (normal), debris, or other cells, which include small clusters, from the patient urine samples. Squamous and columnar cells are commonly found in both sputum and urine and were frequently correctly classified. Small cell clusters were also detected and classified as other cells. Bladder cancer malignant cells are often shed as small clusters, so these detectable clusters may be used in future refinements of the model of the present example for training AI-based classification specifically for bladder cancer.

The instrument did not become clogged at an appreciable rate during sample processing, indicating that processing methods are compatible with the CELL-CT® optical tomography system.

Specific cell counts for each sample are provided in Table 3. All available sample was used in samples designated as exhausted (“Ex”). Object count refers to the total number of objects detected and imaged, whether cells, clusters, or debris. Normal by 2D refers to the number of cells designated as objects other than abnormal cells (i.e. a normal urothelial cell, clusters, debris) by the 2D AI-based classification. Among the objects referred for 3D imaging, the number classified as squamous (“Squ”), columnar (“Col”), and an abnormal cell (“Pos”) are indicated. The number of cells for which the 3D images were off too poor quality for classification is also indicated. This number is expected to be lower with AI-based classification trained to detect bladder cancer. Repeat cells refers to cells detected and imaged at least twice. Repeat cells are removed from cell counts. Positive rate (“Pos rate”) was calculated by dividing the positive cell count (reflecting abnormal cells) by the total object count (reflecting cells and non-cells).

TABLE 3
Cell Counts Per Sample

| | | | | | Object Counts by 3D | | | | | |
|---|---|---|---|---|---|---|---|---|---|---|
| | Cytological | | Object | Normal | Poor | | | | | Pos rate |
| ID | Diagnosis | Ex. | Count | by 2D | Quality | Repeat | Squ | Col | Pos | (%) |
| N17- | Negative | N | 26382 | 16423 | 2824 | 183 | 953 | 67 | 56 | 0.212 |
| 5933 | | | | | | | | | | |
| N17- | Negative | Y | 1632 | 1057 | 116 | 4 | 0 | 60 | 0 | 0.551 |
| 5971 | | | | | | | | | | |
| N17- | Negative | Y | 21463 | 16051 | 1569 | 109 | 622 | 49 | 28 | 0.130 |
| 6000 | | | | | | | | | | |
| N17- | Negative | N | 46065 | 31137 | 6326 | 362 | 1964 | 226 | 224 | 0.486 |
| 6001 | | | | | | | | | | |
| N17- | Negative | Y | 0 | 0 | 0 | 0 | 0 | 0 | 0 | NA |
| 6009 | | | | | | | | | | |
| N17- | Negative | N | 37422 | 25278 | 1302 | 204 | 118 | 114 | 4 | 0.011 |
| 6039 | | | | | | | | | | |
| N17- | Negative | Y | 10207 | 8078 | 538 | 5 | 11 | 9 | 104 | 1.019 |
| 6066 | | | | | | | | | | |
| N17- | Negative | Y | 1074 | 741 | 52 | 0 | 1 | 8 | 66 | 6.145 |
| 6068 | | | | | | | | | | |
| N17- | Negative | Y | 17547 | 11699 | 1207 | 63 | 179 | 161 | 447 | 2.547 |
| 6070 | | | | | | | | | | |
| N17- | Atypical - | N | 20544 | 11069 | 897 | 579 | 89 | 130 | 747 | 3.636 |
| 5884 | CA | | | | | | | | | |
| N17- | Atypical - | Y | 11885 | 7210 | 458 | 150 | 207 | 99 | 152 | 1.279 |
| 5885 | CA | | | | | | | | | |
| N17- | Atypical - | N | 13598 | 8367 | 899 | 152 | 798 | 55 | 217 | 1.596 |
| 5937 | AACA | | | | | | | | | |
| N17- | Atypical - | N | 18784 | 11856 | 1925 | 526 | 11 | 282 | 77 | 0.410 |
| 5942 | AACA | | | | | | | | | |
| N17- | Atypical - | Y | 8287 | 4852 | 369 | 3 | 13 | 108 | 779 | 9.400 |
| 5949 | AACA | | | | | | | | | |
| N17- | Atypical - | Y | 12280 | 6705 | 936 | 149 | 40 | 174 | 1162 | 9.463 |
| 5989 | AACA | | | | | | | | | |
| N17- | Atypical - | Y | 8406 | 4837 | 399 | 20 | 37 | 96 | 753 | 8.958 |
| 6071 | AACA | | | | | | | | | |
| N17- | PFMC - | N | 15682 | 7401 | 1058 | 348 | 37 | 167 | 2059 | 13.130 |
| 5941 | HGUC | | | | | | | | | |
| N17- | PFMC - | Y | 4071 | 1282 | 140 | 16 | 8 | 46 | 236 | 5.797 |
| 6044 | HGUC | | | | | | | | | |

Overall, eleven of eighteen specimens were completely exhausted, many prior to processing of a substantial number of object, which may slightly impair the usefulness of these specimens in evaluating ability to detect bladder cancer. Substantial data was collected for non-exhausted specimens, however, and for some exhausted specimens with high object counts, producing stable positive rate values.

Repeat cell rates were on average only 1.1% of cells, which is a typical value for sputum processing as well. This indicates that cell progression through the optical tomography system and imaging worked generally as expected.

7.8% of cells resulted in poor quality 3D images, which typically exhibit streaking or star artifacts and indicate that the object was too small or too large and, therefore, outside of the size range for which successful 3D image construction is possible. Objects that are too large are typically normal squamous cells, cell clusters, or object clusters. The proportion of poor quality images detected is similar to that for sputum processing, indicating that the imaging and classification worked generally as expected.

The classifiers to identify squamous and columnar were set to unusually high specificity to ensure very accurate positive identification of such cells. This resulted in squamous and columnar cell counts that did not provide an accurate reflection of the prevalence of these cell types in the urine samples. Specificity was set in this manner in this example, however, because squamous cells are used as a basis for automated assessment of proper staining. The ability to detect squamous cells, with at high specificity settings, indicates that the staining protocols used were sufficient to reliably stain cells in urine samples. In AI-based cell classification trained for bladder cells, thresholds for accurate detection of squamous and columnar cells may be determined and used to ensure accurate cell counts. Accurate cell counts for these cell types may be particularly important in methods where the pre-selected threshold number of cells required to be images reflects the combined cell counts of normal urothelial cells, squamous cells, and columnar cell.

Samples from patients with non-cancerous urological diseases or disorders did not affect the positive rate, indicating that results are expected to be accurate for bladder cancer even in patients with other urological disease or disorders.

On average, 61% of a sample was classified as a normal urothelial cell by 2D AI-based classification, indicating that approximately 39% of the total number of cells were 3D imaged and classified by 3D AI-based classification. Similar proportions are expected using AI-based classification trained to detect bladder cancer. In particular, between 50% and 70% of cells in most samples are expected to be excluded from 3D classification in a system trained to detect bladder cancer.

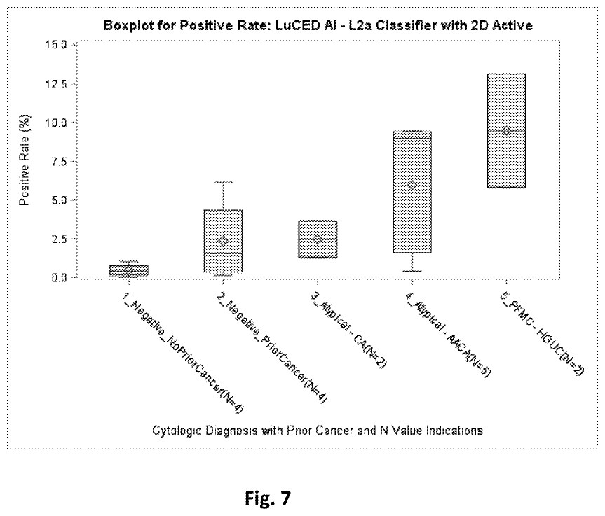

Cytological diagnosis was used to group patients and data was analyzed by sample group. In particular, patients were grouped as 1) negative cytological diagnosis and no prior bladder cancer (n=4); 2) negative cytological diagnosis and prior bladder cancer (n=4); 3) atypical—CA cytological diagnosis (n=2); 4) atypical—AACA cytological diagnosis (n=5); 5) PFMC-HGUC cytological diagnosis (n=2). A boxplot of positive rate per patient group is presented in FIG. 7.

As these results indicate, the positive rate for patients who were determined to be negative for abnormal cells using cytological analysis was low for both patients with no history of cancer and those who previously had bladder cancer, indicating that the assay can reliably exclude patients without abnormal bladder cells. The positive rate for patients with a history of bladder cancer was slightly higher, indicating that some small abnormalities likely persist in such patients. As a result, prior history of bladder cancer may be a relevant factor in analysis of cells by the AI-based cell classifier or of the assay results as a whole for diagnostic purposes.

In addition, the very low positive rate observed in patients without bladder cancer indicates that very few cells in urine have abnormal features. This is a significant difference from sputum, which contains macrophages, repair cells, reactive cells, and degenerated cells in appreciable numbers and, therefore, has a higher positive rate in patients without lung cancer.

The results also indicate that the assay can reliably distinguish patients with urothelial cell atypias from those with no cellular abnormalities and from those with malignant cells. In particular, the positive rate for patients with cells exhibiting atypia and AACA or CA was higher than the positive rate for patients without cancer and lower than the positive rate for patients with malignant cells.

CA typically progresses to HGUC, while AACA typically progresses to LGUC. Accordingly, accurate detection of cells with atypia and CA is of particular clinical significance because of the substantially increased monitoring that may be warranted in such cases.

The positive rate for patients with HGUC was significantly higher, indicating that a substantial number of malignant cells were identified. Patients with LGUC or less progressed GHUC may exhibit lower positive rates that are still higher than those of patients with cellular atypias.

Although kidney cancer cells may also be present in urine, but are expected to be at sufficiently low levels to not significantly increase the positive rate.

Variance of positive rate was very low between samples from patients without bladder cancer or cellular abnormalities and no history of bladder cancer. This provides further evidence that urothelial cells are quite uniform in normal patients. Thus detectable uniformity of normal urothelial cells is expected to also aid in the accurate detection of abnormal cells. Patient factors might explain some of the differences in normal to abnormal group variance. In the data used in the present example, normal groups have multiple patients and low variance, which tends to deflate the patient-patient factors as an explanation for variance. In some embodiments, increasing intra-patient variance as determined by repeated analysis might be an early sign of progression.

Overall, the positive rates appear to support diagnostic results including three groups defined by positive rate thresholds T1 and T2:
Group I: positive rate<T1—Patients with very low risk of bladder cancer or no higher than normal risk
Group II: T1≤positive rate<T2—Patients with a high risk of bladder cancer, likely meriting increased monitoring
Group III: positive rate≤T2—Patients with bladder cancer.

More stratified diagnostic results may be provided using AI-based classification trained using bladder cancer and urine samples.

In addition to establishing that AI-based classification combined with an optical tomographic system may be used to detect bladder cancer and high risk of developing bladder cancer, the data in this example also provides an estimate of the number of cells required to be processed in order to obtain accurate results.

The data presented in Table 3 and FIG. 7 establishes that the positive rate is stratified based on the patient clinical condition. The middle ground in the positive rate between negative cases without cancer recurrence and cancer is about 2%. This middle ground group is between patients in the negative group and no history of cancer and those with verified abnormalities. Data from patients in the negative group, but with a history of cancer was excluded to give a sense for how a screening program would play out in practice, where most (>95%) of the screening population would have no history of cancer. The data shows that almost perfect discrimination between N/no cancer history and abnormal can be managed. In practice, patients with a history of cancer would likely be user special management by a clinician, and would not be referred for testing according to the present disclosure, or the test results would likely not be relied upon as a sole indicator of cancer status for such a patient.

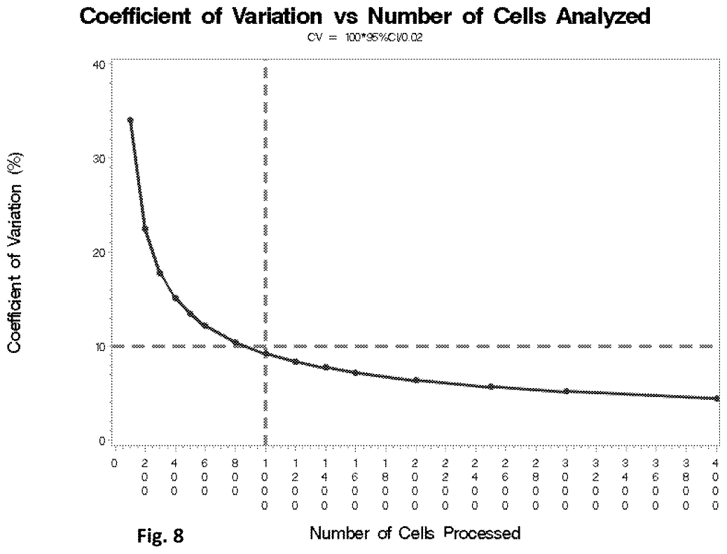

Therefore, the positive rate ideally achieves stability for a measure of 2%. Stability was assessed through use of the binomial theorem to assess variation in the ratio given a certain N value for the denominator. The JavaState 4 binomial confidence interval calculator was used to assess the 95% confidence interval in a 2% ratio for various numbers of objects imaged ranging from 100 to 4000. The 95% confidence interval was divided by 0.02 and multiplied by 100% for a percentage, thus producing the 95% confidence interval coefficient of variation (CV). FIG. 8 shows the relationship between the CV and number of objects processed. Although an acceptable CV may be different in some more refined assays using AI-based classification trained for bladder caner and urine samples, depending on what CV is associated with a pre-selected diagnostic accuracy, 10% was used as a threshold in this example. As illustrated in FIG. 8, 1000 objects were required to be imaged to achieve satisfactory results. Thus, the threshold number of objects for this assay would be 1000.

The threshold number of object may also be adjusted to ensure that an acceptable percentage of samples will meet this threshold and the assay will not frequently fail to provide diagnostic information.

Given that approximately 40% of cells in this example were referred for 3D imaging the processing time required for 1000 objects would have been approximately 30 minutes. Total processing time for 1000 objects, allowing for sample loading and removal, is expected to be approximately 1 hour.

### Example 3

### Detection of Infection

Specimen N17-6039 was negative for abnormal cells as determined by cytological diagnosis. However, a substantial number of neutrophils were identified by 3D AI-based classification of this sample. This indicates that the method was able to detect urinary tract infection. Using analysis similar to that of Example 2, a threshold number of objects and a white blood cell positive rate could be developed for use in detection of infections. Due to variability of the numbers of white blood cells in urine depending on the severity of infection, a threshold number of normal urothelial cells and squamous cells might be used in place of a threshold object count.

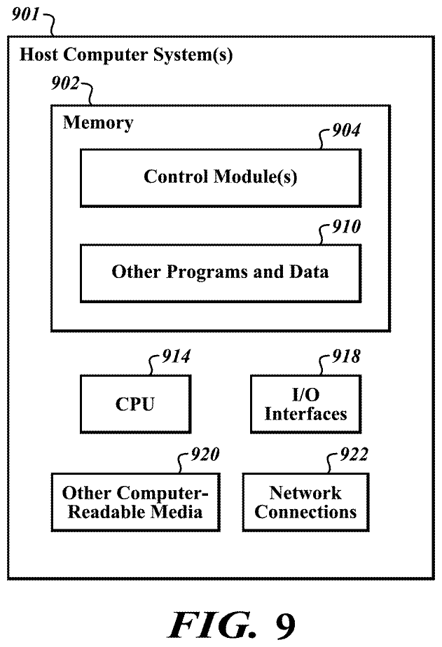

FIG. 9 shows a system diagram that describes an example implementation of a computing system(s) for implementing embodiments described herein. The functionality described herein for a system for a method for providing equivalent services that can be implemented either on dedicated hardware, as a software instance running on dedicated hardware, or as a virtualized function instantiated on an appropriate platform, e.g., a cloud infrastructure. In some embodiments, such functionality may be completely software-based and designed as cloud-native, meaning that they are agnostic to the underlying cloud infrastructure, allowing higher deployment agility and flexibility.

In particular, shown is example host computer system(s) 901. For example, such computer system(s) 901 may represent those in various data centers and/or described herein that host the functions, components, microservices and other aspects described herein to implement a method for providing equivalent services to user devices across multiple participating telecommunication networks. In some embodiments, one or more special-purpose computing systems may be used to implement the functionality described herein. Accordingly, various embodiments described herein may be implemented in software, hardware, firmware, or in some combination thereof. Host computer system(s) 901 may include memory 902, one or more central processing units (CPUs) 914, I/O interfaces 918, other computer-readable media 920, and network connections 922.

Memory 902 may include one or more various types of non-volatile and/or volatile storage technologies. Examples of memory 902 may include, but are not limited to, flash memory, hard disk drives, optical drives, solid-state drives, various types of random-access memory (RAM), various types of read-only memory (ROM), other computer-readable storage media (also referred to as processor-readable storage media), or the like, or any combination thereof. Memory 902 may be utilized to store information, including computer-readable instructions that are utilized by CPU 914 to perform actions, including those of embodiments described herein.

Memory 902 may have stored thereon control module(s) 904. The control module(s) 904 may be configured to implement and/or perform some or all of the functions of the systems, components and modules described herein for a method for providing equivalent services to user devices across multiple participating telecommunication networks. Memory 902 may also store other programs and data 910, which may include rules, databases, application programming interfaces (APIs), software platforms, cloud computing service software, network management software, network orchestrator software, network functions (NF), AI or ML programs or models to perform the functionality described herein, user interfaces, operating systems, other network management functions, other NFs, and the like.

Network connections 922 are configured to communicate with other computing devices to facilitate the functionality described herein. In various embodiments, the network connections 922 include transmitters and receivers (not illustrated), cellular telecommunication network equipment and interfaces, and/or other computer network equipment and interfaces to send and receive data as described herein, such as to send and receive instructions, commands and data to implement the processes described herein. I/O interfaces 918 may include a video interface, other data input or output interfaces, or the like. Other computer-readable media 920 may include other types of stationary or removable computer-readable media, such as removable flash drives, external hard drives, or the like.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Throughout the specification, claims, and drawings, the following terms take the meaning explicitly associated herein, unless the context clearly dictates otherwise. The term “herein” refers to the specification, claims, and drawings associated with the current application. The phrases “in one embodiment,” “in another embodiment,” “in various embodiments,” “in some embodiments,” “in other embodiments,” and other variations thereof refer to one or more features, structures, functions, limitations, or characteristics of the present disclosure, and are not limited to the same or different embodiments unless the context clearly dictates otherwise. As used herein, the term “or” is an inclusive “or” operator, and is equivalent to the phrases “A or B, or both” or “A or B or C, or any combination thereof,” and lists with additional elements are similarly treated. The term “based on” is not exclusive and allows for being based on additional features, functions, aspects, or limitations not described unless the context clearly dictates otherwise. In addition, throughout the specification, the meaning of “a,” “an,” and “the” include singular and plural references.

The following description, along with the accompanying drawings, sets forth certain specific details to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components associated with the environment of the present disclosure have not been shown or described to avoid unnecessarily obscuring descriptions of the embodiments. Additionally, the various embodiments may be methods, systems, or devices.

1. An artificial intelligence (AI)-based urine cell detection method comprising:
generating, for a plurality of objects in a urine sample and by an optical tomography system, a 3D image of an object from the urine sample; and
evaluating the 3D image using 3D AI-based classification to determine if the object is an abnormal cell or an object other than an abnormal cell.

2. The method of claim 1, wherein, prior to generating the 3D image, the method comprises:
generating, by the optical tomography system, a 2D image of the object from the urine sample; and
evaluating the 2D image using 2D AI-based classification to determine if the object is likely an abnormal cell or likely to be an object other than an abnormal cell, wherein the 3D image is generated only if the object is determined to likely be an abnormal cell through 2D classification.

3. The method of claim 1, further comprising repeating imaging and evaluating objects and classifying each object as an abnormal cell or an object other than an abnormal cell for a plurality of objects in the urine sample to generate urine sample data that includes a total object count reflecting the total number of objects classified and an abnormal cell count reflecting the total number of objects determined to be abnormal cells.

4. The method of claim 3, further comprising comparing the total object count to an object count threshold number and imaging and classifying additional objects in the urine sample if the total object count is lower than the total object count threshold number.

5. The method of claim 4, wherein the total object count threshold number is sufficient to ensure a pre-selected accuracy of classification of abnormal cells, and wherein the total object count threshold number is between 900 and 1100.

6. The method of claim 3, wherein the total object count comprises the total number of urothelial cells that are other than abnormal cells or the total number of urothelial cells and squamous cells that are other than abnormal cells.

7. The method of claim 2, wherein classification specificity for abnormal cells for the overall method is calculated as 100%×(1−FPr_3D×FPr_2D), wherein FRP_3D refers to the false positive rate of classification using the 3D image, and FPR_2D refers to the false positive rate of classification using the 2D image.

8. The method of claim 2, wherein the method has a pre-selected 2D AI-based classification sensitivity value for one or more of abnormal cells of at least 90% and objects other than abnormal cells of at least 90%.

9. The method of claim 1, wherein abnormal cell classifications are reflected in an abnormal cell count, which correlates with a patient who provided the urine sample has urogenital cancer, is at a higher than normal risk for having urogenital cancer, is it at an average or lower risk for having urogenital cancer, has a risk of having urogenital cancer above a pre-selected threshold, or has a risk of having urogenital cancer below a pre-selected threshold, and wherein the urogenital cancer is kidney cancer, bladder cancer, urethral cancer, or prostate cancer.

10. The method of claim 1, wherein the urine sample comprises abnormal cells, urothelial cells other than abnormal cells, squamous cells, columnar cells, white blood cells, debris, cell clusters, and any combinations thereof.

11. The method of claim 1, further comprising, prior to generating any image: pre-processing the urine sample to stain the plurality of cells with an agent that facilitates generating the 2D image, if generated, evaluating the 2D image using 2D AI-based classification, if so classified, generating the 3D image, or evaluating the 3D image using 3D AI-based classification.

12. The method of claim 1, further comprising, prior to generating any image:
embedding the urine sample in an optical medium and injecting the optical medium with embedded sample into a capillary tube; and
loading the capillary tube into the optical tomography system so that the capillary tube is between an illumination source and objective lens of the optical tomography system.

13. The method of claim 2, wherein generating, by the optical tomography system, the 2D image of the cell comprises the optical tomography system sweeping a focal plane of the optical tomography system in 1 μm steps across a single cell to generate a single-plane 2D image of the single cell at each step, compiling a plurality of single-plane 2D images of the single cell, and filtering the set of single-plane 2D images of the single cell to generate a representative 2D image of the cell that is evaluated.

14. The method of claim 2, wherein evaluating the 2D image using 2D AI-based classification comprises determining values for a plurality of 2D image cell feature measurements, and wherein the cell feature measurements comprise object shape features, cell-shape features, cytoplasm features, cell nucleoli features, distribution of chromatin, nuclear-size features, nuclear-texture features, other morphometric elements, or any combination thereof.

15. The method of claim 1, wherein evaluating the 3D image using 3D AI-based classification comprises determining values for a plurality of 3D image cell feature measurements, and wherein the cell feature measurements comprise object shape features, cell-shape features, cytoplasm features, cell nucleoli features, distribution of chromatin, nuclear-size features, nuclear-texture features, other morphometric elements, or any combination thereof.

16. The method of claim 1, further comprising:
determining if the object is a red blood cell (RBC) and performing all steps as if the RBC were the abnormal cell, and
using a total RBC count to determine whether the patient has urogenital cancer, is at a higher than normal risk for having urogenital cancer, is it at an average or lower risk for having urogenital cancer, has a risk of having urogenital cancer above a pre-selected threshold, or has a risk of having urogenital cancer below a pre-selected threshold.

17. The method of claim 1, further comprising determining if the object is a white blood cell (WBC) or a specific type of WBC and performing all steps as if the WBC or specific type of WBC were the abnormal cell, and one or more of:
using the total WBC or type of WBC count to determine whether the patient has urogenital cancer, is at a higher than normal risk for having urogenital cancer, is it at an average or lower risk for having urogenital cancer, has a risk of having urogenital cancer above a pre-selected threshold, or has a risk of having urogenital cancer below a pre-selected threshold.
using the total WBC count or type of WBC count to determine whether the patient has a urinary tract infection.
using the total WBC count or type of WBC count to determine whether the patient is responding to immune checkpoint inhibitor therapy for urogenital cancer.

18. The method of claim 1, further comprising determining if the object is a uric acid crystal and performing all steps as if the uric acid crystal were the abnormal cell, and one or more of:
using a total uric acid crystal count to determine whether the patient has urogenital cancer, is at a higher than normal risk for having urogenital cancer, is it at an average or lower risk for having urogenital cancer, has a risk of having urogenital cancer above a pre-selected threshold, or has a risk of having urogenital cancer below a pre-selected threshold.
using a total uric acid crystal count to determine if the patient has a kidney stone.

19. The method of claim 1, further comprising:
determining if the object is a cell cluster and performing all steps as if the cell cluster were the abnormal cell; and
using a total cell cluster count to determine whether the patient has urogenital cancer, is at a higher than normal risk for having urogenital cancer, is at an average or lower risk for having urogenital cancer, has a risk of having urogenital cancer above a pre-selected threshold, or has a risk of having urogenital cancer below a pre-selected threshold.

20. An object classification system comprising an optical tomography system operable to:
generate a 3D image of an object in a urine sample; and
evaluate the 3D image using 3D AI-based classification to determine if the object is an abnormal cell or an object other than an abnormal cell.

21. The object classification system of claim 20, wherein the optical tomography system is further operable to:
generate a 2D image of the object; and
evaluate the 2D image using 2D AI-based classification to determine if the object is likely an abnormal cell or likely an object other than an abnormal cell.

22. An object classification system comprising an optical tomography system operable to perform the method of claim 1.
